(19) United States
(12) Patent Application Publication
(10) Pub. No.: US 2023/0317246 A1
(43) Pub. Date: Oct. 5, 2023

(54) SYSTEM AND METHOD FOR FACILITATING MENTAL HEALTH ASSESSMENT AND ENHANCING MENTAL HEALTH VIA FACIAL RECOGNITION

(71) Applicant: BeMe Health, Inc., Marblehead, MA (US)

(72) Inventors: Nivaaz K Dhillon, Palmer, PR (US); Ruth M Tessler, Surfside, FL (US); Hazuri K Dhillon, Palmer, PR (US); Prabhanjan Gurumohan, Palo Alto, CA (US); Nicoletta Tessler, Surfside, FL (US); Mandeep Dhillon, Palmer, PR (US); Neha Chaudhary, San Francisco, CA (US); Elisha Ferguson, Brooklyn, NY (US); Danielle Ramos-Larios, Oakland, CA (US)

(73) Assignee: BeMe Health, Inc., Marblehead, MA (US)

(21) Appl. No.: 18/148,804

(22) Filed: Dec. 30, 2022

Related U.S. Application Data
(60) Provisional application No. 63/326,646, filed on Apr. 1, 2022.

(51) Int. Cl. G16H 20/70; G16H 50/20; G16H 40/67; A61B 5/16

(52) U.S. Cl. CPC G16H 20/70; G16H 50/20; G16H 40/67; A61B 5/165

(57) ABSTRACT

A system for facilitating mental health assessment and enhancing mental health via facial recognition and content associated with other physical attributes is provided. In particular, the system receives content associated with physical attribute of a user from at least one sensor and receives self-assessed emotional states from the user. The system extracts features from the content and determines, based on the content and by utilizing artificial intelligence models, predicted emotional states of the user by comparing the features to training information utilized to train the artificial intelligence models. The system proceeds to identify content to deliver to the user and recommend activities for the user to enhance or maintain the self-assessed emotional states, the predicted emotional states, or a combination thereof. The system also tracks emotional state changes over time based on the user interacting with the content and participating in activities and dynamically adjusts the contents and recommendations accordingly.

### CROSS REFERENCE TO RELATED APPLICATIONS

The present application claims priority to and the benefit of U.S. Provisional Application No. 63/326,646, filed on Apr. 1, 2022, which is hereby incorporated by reference in the present disclosure in its entirety.

### FIELD OF THE INVENTION

The present application relates to mental health enhancement technologies, data analytics technologies, facial recognition and analysis technologies, biometric recognition and analysis technologies, artificial intelligence technologies, machine learning technologies, cloud-computing technologies, interactive technologies, and, more particularly, to a system and method for facilitating mental health assessment and enhancing mental health via facial recognition.

### BACKGROUND

In today's society, effectively identifying an individual's emotional or mental health and implementing measures to improve or maintain the individual's emotional health has become increasingly important. Emotional and mental health may be affected by a plurality of factors, including, but not limited to, physical health, physical and substance abuse, self-esteem, isolation, environmental factors, nutrition, relationships, activity levels, genetic factors, other factors, or a combination thereof. Additionally, while the ever-increasing intertwining of daily life with various forms of technology has led to numerous efficiencies, conveniences, and productivity increases, such intertwining has resulted in negative impacts on emotional and mental health. For example, the technological abilities to remotely communicate, participate in remote meetings, engage in social media, and participate in other forms of technology-based interaction have resulted in fewer in-person interactions, reduced emotional intelligence, among other negative effects. The foregoing effects are especially present in today's teenager population, which is even more reliant on technology-based communication than other populations.

With regard to the teenager population, teenagers often lack emotional intelligence due to lack of experience and may not have the necessary tools to understand and express their emotions properly. For example, while teenagers might believe that they are expressing a particular emotion, teenagers may actually be feeling an entirely different or related emotion and may not be able to identify the different or related emotion on their own. Additionally, teenagers may resort to being dishonest about the way that they truly feel. For example, while teenagers may say that they are feeling a certain way, in reality, they may exhibit completely different emotions. Being able to express oneself is a function of the prefrontal cortex, which, for a teenager, is still under development. As a result, teenagers often use the amygdala for decision making and solving problems. The amygdala is associated with instincts, emotions, impulsiveness, and aggression. As a result, it may be hard to readily understand teens' emotions.

Moreover, teenagers' lives are complex both inside and out. For example, teenagers experience tremendous internal hormonal changes that both produce and manage teenagers' emotional lives. From the external world, teenagers experience dramatic and dynamic shifts in the structure and importance of critical social interactions, including those with peers, romantic interests, and parents, and a range of new experiences and competing societal demands. The collision of a teen's inside and out experiences can impact emotions and behaviors that are at time overwhelming, confusing, and hard to manage. At the same time, teens are also avid users of technology, which as discussed above, impact emotional and mental health.

Typically, individuals may seek assistance with improving their mental and emotional health by consulting with mental health professionals, such as therapists, psychiatrists, and psychologists. Such mental health professionals typically assess and evaluate the mental health of individuals during a therapy session at a clinical or office setting. Assessments relating to mental health may be made by the professionals based on questions posed to individuals, observations relating to responses provided by individuals, and analyzing the responses and observations based on their mental health expertise. Certain assessment tests have been widely used by mental health professionals to screen for mental health conditions and track changes in symptom severity over time. Recently, such professionals and technology companies have employed the use of software applications for content delivery and telemedicine to connect mental health patients to their care provider.

Nevertheless, despite the foregoing, there remains room for substantial enhancements to existing technologies and processes maintain and enhance emotional health, mental health, and overall well-being. While currently existing technologies provide for various benefits, such technologies still come with various drawbacks and inefficiencies. For example, currently existing processes and technologies often do not detect or prevent negative mental or emotional health issues early enough or fast enough to have meaningful impact, especially when it comes to reducing potential negative consequences, such as lasting depression or suicide. While currently existing processes may have short-term effectiveness on a case-by-case basis, existing technologies often fail to have a lasting effect on stabilizing or improving mental or emotional health. Moreover, existing technologies fail to take advantage of artificial intelligence technologies that may assist a system in adapting to changing mental health needs. Based on the foregoing, new technologies may be provided that facilitate improvements to mental and emotional health, promote preventative behaviors, and provide guidance for individuals with struggles, such as those outside traditional behavioral health issues, including those needing to adhere to physical healthcare services. Such enhancements and improvements to processes and technologies may provide for enhanced mental health wellness, increased individual satisfaction with mental wellness programs, and, ultimately, improved mental and emotional health for individuals.

### SUMMARY

A system and accompanying methods for facilitating mental health assessment and enhancing mental health via facial recognition and sensor data associated with physical attributes and expressions are disclosed. In particular, the system and methods utilize devices and applications in combination with artificial intelligence models (e.g., machine learning models) to provide a unique and different ability to assess, evaluate, and improve the mental and emotional health of individuals. In certain embodiments, the system and methods may monitor and track individuals' emotions and determine mental and emotional states for the individuals with a high probability of accuracy. The system and methods may identify content for the individuals to experience and recommend actions that will assist the individuals to enhance or maintain mental and emotional health and to be more logical in dealing with mental, emotional, and developmental issues.

In certain embodiments, the system and methods may provide an application serving as a digital companion to help individuals enhance or maintain their mental and emotional health. For example, for teens, the application may help teens through teenage period of their lives, which involves increased levels of emotionality and substantially more psychopathological levels of dysfunctional affective experiences. In certain embodiments, the system and methods may also enable individuals to learn to manage their emotional reactions. Still further, the system and methods may enable individuals to understand psychological factors that interface with individual's emotional life that are contributing to their experiences.

In certain embodiments, the system and methods may incorporate the use of algorithms that track various activities performed and/or participated in by an individual that helps improve or manage mood, health, and relationships. In certain embodiments, signals including data associated with such activities may be digital and anatomical, and may be used to score the user and, in turn, the score may be utilized to recommend activities that may assist the individual in overcoming and/or improving a mental and/or emotional health issue. The system and methods may analyze sensor data, such as images of facial features taken at a certain point in time to serve as mood indicators and may predict emotional and/or mental states of individuals based on the sensor data. In certain embodiments, artificial intelligence models and/or machine learning models may be trained to correlate features and/or vectors extracted from sensor data to emotions, moods, and the like. In certain embodiments, the system and methods may also receive self-assessments of emotional and mental states from individuals and determine whether the self-assessments are accurate based on comparing the self-assessments to the emotional and/or mental states predicted from the sensor data. Based on the determined emotional and/or mental states of the individuals, the system and methods may generate and/or identify content to deliver to the user to maintain and/or enhance the individuals' emotional and/or mental states and generate recommendations for activities for the user to participate in. Compliance with performing the activities and experiencing the content may be tracked and adjustments to emotional and/or mental health may be monitored. Based on the tracking and monitoring, the artificial intelligence and/or machine learning models may be updated to enhance predictive capabilities, identification and generation of content, and generation of recommendation of activities to perform over time.

Based on at least the foregoing capabilities, the system and methods may provide a dynamic system of prediction that uniquely provides for significantly greater understanding of an individual's mental and emotional health. Such a system significantly broadens the number of signals utilized to assess, evaluate, and/or improve mental health, dynamically uses data to refine and calibrate mental health recommendations, and, ultimately, creates implementable and personalized functionality that assists an individual in improving mental and emotional health through recommended content, care activities, coaching, community, crises resources, therapy referrals, and other resources.

In one embodiment, a system facilitating mental health assessment and enhancing mental health via facial recognition and content associated with physical attributes and expressions is provided. The system may include a memory that stores instructions and a processor that executes the instructions to perform various operations of the system. The system may perform an operation that includes receiving, via a device, content associated with one or more physical attributes, one or more expressions, or a combination thereof, of a user. In certain embodiments, the content associated with the one or more physical attributes, the one or more expressions, or a combination thereof, may be obtained via one or more sensors. In certain embodiments, the system may perform an operation that includes receiving, from the user, one or more self-assessed emotional states currently being experienced by the user. In certain embodiments, the system may perform an operation that includes extracting, by utilizing at least one artificial intelligence model (and/or machine learning model), one or more features from the content. Based on the content and by utilizing at least one artificial intelligence model, the system, in certain embodiments, may determine one or more predicted emotional states of the user, wherein the at least one predicted emotional states of the user based on comparing the one or more features extracted from the content to training information utilized to train the at least one artificial intelligence model. In certain embodiments, the system may perform an operation that includes identifying, by utilizing the at least one artificial intelligence model, content to deliver to the user to enhance or maintain the one or more self-assessed emotional states, the one or more predicted emotional states, or a combination thereof. In certain embodiments, the system may perform an operation that includes providing, to the device, access to the content to facilitate enhancement or maintenance of the one or more self-assessed emotional states, the one or more predicted emotional states, or a combination thereof.

In another embodiment, a method for facilitating mental health assessment and enhancing mental health via facial recognition and content associated with physical attributes and expressions is disclosed. The method may include a memory that stores instructions and a processor that executes the instructions to perform the functionality of the method. In certain embodiments, the method may include receiving, such as via an application executing on a device, content associated with at least one physical attribute of a user, at least one expression, or a combination thereof. In certain embodiments, the content associated with the at least one physical attribute, the at least one expression, or a combination thereof, may be obtained via one or more sensors. In certain embodiments, the method may include receiving, from the user via the application, one or more self-assessed emotional states currently being experienced by the user. In certain embodiments, the method may include extracting, by utilizing at least one artificial intelligence model (and/or machine learning model), one or more features from the content. In certain embodiments, the method may include determining, based on the content and by utilizing at least one artificial intelligence model (and/or machine learning model), at least one predicted emotional state of the user. In certain embodiments, the at least one predicted emotional state of the user may be determined based on comparing the one or more features extracted from the content to training information utilized to train the at least one artificial intelligence model (and/or machine learning model). In certain embodiments, the method may include generating, by utilizing the at least one artificial intelligence model (and/or machine learning model), content to deliver to the user to enhance or maintain the at least one self-assessed emotional state, the at least one predicted emotional state, or a combination thereof. In certain embodiments, the method may include providing, to the device, access to the content to facilitate enhancement or maintenance of the at least one self-assessed emotional state, the at least one predicted emotional state, or a combination thereof.

According to yet another embodiment, a computer-readable device comprising instructions, which, when loaded and executed by a processor cause the processor to perform operations, the operations comprising: receiving, via an application executing on a device, content associated with at least one physical attribute of a user, at least one expression, or a combination thereof, wherein the content associated with the at least one physical attribute, the at least one expression, or a combination thereof, is obtained via at least one sensor associated with the device; receiving, from the user via the application, at least one self-assessed emotional state currently being experienced by the user; extracting, by utilizing at least one artificial intelligence model (and/or machine learning model), at least one feature from the content; determining, based on the content and by utilizing at least one artificial intelligence model (and/or machine learning model), at least one predicted emotional state of the user, wherein the at least one predicted emotional state of the user is determined based on comparing the at least one feature extracted from the content to training information utilized to train the at least one artificial intelligence model (and/or machine learning model); generating, by utilizing the at least one artificial intelligence model (and/or machine learning model), content to deliver to the user to enhance or maintain the at least one self-assessed emotional state, the at least one predicted emotional state, or a combination thereof; and providing, to the device, access to the content to facilitate enhancement or maintenance of the at least one self-assessed emotional state, the at least one predicted emotional state, or a combination thereof.

These and other features of the systems and methods for facilitating mental health assessment and enhancing mental health via facial recognition and content associated with physical attributes and expressions are described in the following detailed description, drawings, and appended claims.

### BRIEF DESCRIPTION OF THE DRAWINGS

FIG. 1 is a schematic diagram of a system for facilitating mental health assessment and enhancing mental health via facial recognition and content associated with physical attributes and expressions according to embodiments of the present disclosure.

FIG. 2 is an exemplary illustration of various information, components, and aspects of the system of FIG. 1 according to embodiments of the present disclosure.

FIG. 3 illustrates an exemplary user interface of an application supporting the functionality of the system of FIG. 1 that enables a user to record content of the user according to embodiments of the present disclosure.

FIG. 4 illustrates an exemplary user interface of an application that features an exemplary image taken by a user according to embodiments of the present disclosure.

FIG. 5 illustrates an exemplary user interface of an application illustrating the ability to enable a user to self-assess the user's emotional state, mental state, and mood according to embodiments of the present disclosure.

FIG. 6 illustrates an exemplary user interface illustrating tagging of the exemplary image of FIG. 4 with self-assessed emotional states, mental states, and/or moods according to embodiments of the present disclosure.

FIG. 7 illustrates an exemplary user interface illustrating a capability of being able to share the tagged image of FIG. 6 according to embodiments of the present disclosure.

FIG. 8 illustrates an exemplary user interface illustrating various information associated with a user's emotional states and confidence levels associated with the user's emotional states according to embodiments of the present disclosure.

FIG. 9 is a flow diagram illustrating a sample method for facilitating mental health assessment and enhancing mental health via facial recognition and content associated with physical attributes and expressions according to embodiments of the present disclosure.

FIG. 10 is a schematic diagram of a machine in the form of a computer system within which a set of instructions, when executed, may cause the machine facilitate mental health assessment and enhance mental health via facial recognition and content associated with physical attributes and expressions according to embodiments of the present disclosure.

### DETAILED DESCRIPTION

A system 100 and accompanying methods (e.g., method 900) for facilitating mental health assessment and enhancing mental health via facial recognition are disclosed. In certain embodiments, the system 100 and methods may facilitate mental health assessment and enhancement of mental health based on content associated with other physical attributes of a user, expressions of a user, or a combination thereof. In particular, the system 100 and methods utilize devices and applications in combination with artificial intelligence models (e.g., machine learning models) to provide a unique and different ability to assess, evaluate, and/or enhance the mental health statuses of individuals interacting with the system 100. In certain embodiments, the system 100 and methods provide processes that can effectively track a user's emotions with a high probability of correctness and generate recommendations for actions to perform that would assist the user in maintaining or enhancing their mental health, emotional health, or a combination thereof. In certain embodiments, an application supporting the system 100 and methods may enable a user to perform activities that are categorized both on wellness categories and clinical categories. Based on the user's actions, a series of next actions to perform may be recommended or suggested based on the operative functionality of the system and by utilizing techniques described in U.S. Provisional Application No. 63/326,646, filed on Apr. 1, 2022, which, as indicated above, is hereby incorporated by reference in the present disclosure in its entirety.

In certain embodiments, for example, the system 100 and methods may include prompting or enabling users to capture content associated with themselves, which may be analyzed by the system 100 and methods to determine moods, emotional states, mental health states, or a combination thereof, of the users. For example, the application supporting the functionality of the system 100 may prompt, via a user interface of the application, a user to take any number of pictures (or video, audio, and/or other types of content including sensor data) of herself using a device, such as a smartphone. In certain embodiments, the system 100 and methods may enable the user to self-assess her emotional state, mental state, or a combination thereof, as depicted in the pictures by providing the ability to digitally tag the pictures with her self-assessments (e.g., self-assessments as words, emojis, avatars, etc.). In certain embodiments, the system 100 and methods may optionally also utilize sensor data from other sensors, such as temperature sensors, motion sensors, and/or other sensors to provide sensor data to facilitate the determination of the user's moods, emotional states, and/or mental states.

In certain embodiments, the system 100 and methods may utilize various artificial intelligence and machine learning techniques to detect the user's moods in the captured content and/or sensor data. For example, the system 100 and methods may utilize convolutional neural networks (e.g., convolutional layers), vision transformers, and/or other machine learning technologies to conduct tasks such as, but not limited to, image classification, image segmentation, content-based image retrieval, object detection, and other computer vision tasks on the content captured that is associated with the user. In certain embodiments, such techniques may be utilized to detect mood indicators (e.g. facial expressions, such as a frown, smile, furrowed eyebrows, squinted eyes, wrinkles on the forehead, etc.) within the content. The artificial intelligence and/or machine learning techniques may detect such mood indicators by comparing the images (or other content and/or sensor data) of the user to training information utilized to train the artificial intelligence and/or machine learning models utilized by the system 100 and methods. In certain embodiments, for example, the models may be trained with information that indicates that a frown corresponds with unhappiness, sadness, and/or depression. If the system 100 detects a frown in the image, the system 100 may determine that the user's emotional state is one of sadness based on the correlation with the training information indicating that a frown in an image is associated with the emotional state of sadness.

In certain embodiments, the system 100 and methods may determine whether the self-assessments made by the user are accurate. For example, the user may have tagged herself as being happy, however, the machine learning models of the system 100 may have analyzed the images of the user taken at the time of tagging and determined (or predicted) that the user is actually sad, anxious, and nervous. In such a scenario, the system 100 may select the predicted emotional state over the self-assessed emotional state. In certain embodiments, the system 100 and methods may select an emotional state for the user that has characteristics of the predicted emotional state and the self-assessed emotional state. In certain embodiments, the determination as to which emotional state to select may be further supplemented and/or finalized based on the user's history (e.g. interactions with the application itself, life events, indication of having gone to therapy sessions, life circumstances, etc.), the user's interactions with content provided by the system 100 or otherwise, the user's performance of recommended activities, the user's characteristics (e.g., demographic information, psychographic information, vitals, physical attributes, location information, etc.), any other information, or a combination thereof.

Once the user's emotional and/or mental state are selected by the system 100 and methods, the system 100 and methods may generate and/or identify content to be delivered to the user that may enhance or maintain the user's emotional state. For example, if the user's emotional state is a happy state, the system 100 may generate a video clip of a person having fun at the beach, which may be presented to the user's device and may have been identified by the system as content that would maintain the user's happy emotional state. As another example, the system 100 and methods may suggest that the user play a video game to keep the user in a happy emotional state. In certain embodiments, in addition to identifying and/or generating content, the system 100 and methods may recommend certain activities to perform to enhance or maintain the user's emotional state. For example, the system 100 and methods may recommend certain activities to perform in a certain sequence, time of day, and/or duration. The system 100 and methods may monitor the user's reactions and/or responses to the content and/or compliance with participation in the recommended activities. In certain embodiments, the system 100 and methods may prompt the user to take new pictures (or other content and/or sensor data) during and/or after experiencing the content and/or performing the activities to detect changes in the emotional state of the user and to determine whether the recommended activities and/or content were effective in maintaining and/or enhancing the user's emotional state. In certain embodiments, the system 100 and methods may utilize the predictions, self-assessments, monitoring, effectiveness, and/or any other information generated and/or analyzed by the system 100 to train the artificial intelligence and machine learning models of the system 100. The training may be utilized to enhance future predictions of emotional states for the user, other users, or a combination thereof.

In certain embodiments, the system 100 and methods may be configured to determine, by utilizing the at least one artificial intelligence and/or machine learning model, whether a deviation between the self-assessed emotional state and the at least one predicted emotional state of the user exists. In certain embodiments, the system 100 and methods may be configured to train the artificial intelligence and/or machine learning model(s) to facilitate a prediction for a future emotional state of the user, another user, or a combination thereof, based on the deviation if the deviation between the self-assessed emotional state and the predicted emotional state of the user is determined to exist. In certain embodiments, the system 100 and methods are configured to determine the predicted emotional state of the user based on identifying a correlation of at least one feature extracted from the content associated with the user with a pattern in the training information corresponding to at least one known emotional state. In certain embodiments, the system 100 and methods may be configured to determine whether the predicted emotional state or the self-assessed emotional state has a higher probability of being the actual emotional state of the user. In certain embodiments, the system 100 and methods may be configured to select the predicted emotional state as the actual emotional state for the user if the predicted emotional state has the higher probability of being the actual emotional state of the user.

In certain embodiments, the system 100 and methods may be configured to determine a score value relating to a mental health of the user based on analyzing a plurality of signals associated with a mood, a mental state, or a combination thereof, associated with the user, interaction data associated with the user, or a combination thereof. In certain embodiments, the system 100 and methods may be configured to determine a deviation between the score value relating to the mental health of the user and the predicted emotional state, the self-assessed emotional state, an actual emotional state, or a combination thereof. In certain embodiments, the system 100 and methods are configured to receive additional information associated with the user to facilitate the determinations of the system 100. For example, the additional information associated with the user may include a plurality of markers associated with the user, including, but not limited to, location information (e.g., user's location and/or device's location), demographic information, psychographic information, life event information, emotional action information, movement information (e.g., the user's movements), health information, audio information, virtual reality information, augmented reality information, time-related information, physical activity information, mental activity information, diet information, experience information, sociocultural information, political information, relationship information, or a combination thereof.

In certain embodiments, the content associated with the physical attributes, expressions, or a combination thereof, of the user may include content image content, video content, audio content, haptic content, vibration content, blood pressure data, sweat data, heart rate data, breath data, breathing data, glucose data, gesture data, motion data, speed data, orientation data, or a combination thereof. In certain embodiments, the video content may include indications of facial expression, at least one facial movement, or a combination thereof. In certain embodiments, the audio content may indicate a rate of speech, a tone of the user, a pitch of the user, a volume of speech of the user, or a combination thereof. In certain embodiments, the system 100 and methods may be configured to combine at least one characteristic of the self-assessed emotional state with at least one characteristic of the predicted emotional state to define at least one actual emotional state of the user. In certain embodiments, the self-assessed emotional state may identify an emotional state of the user as expressed in the content associated with the user (e.g., image, video, and/or other content).

In certain embodiments, the system 100 and methods may include prompting the user to identify the self-assessed emotional state within the content obtained via the sensor associated with device. In certain embodiments, the system 100 and methods may include determining a type of content to deliver to the user to enhance or maintain the self-assessed emotional state, the predicted emotional state, or a combination thereof. In certain embodiments, the system 100 and methods may include providing an option, via the application, to enable the user to provide information reflecting on the self-assessed emotional state, the predicted emotional state, an enhancement the predicted or self-assessed emotional state, or a combination thereof. In certain embodiments, the system 100 and methods may include a recommendation for an activity for the user to perform to facilitate enhancement or maintenance of the self-assessed emotional state, the predicted emotional state, or a combination thereof. In certain embodiments, the system 100 and methods may include requesting the user to generate, for the application, baseline content and identify at least one actual emotional state of the user as represented by the baseline content. For example, the baseline content may be images or video content that definitely indicate a particular mood, emotional state, mental state, or a combination thereof, which may be confirmed by the user at the outset of using the application (e.g. registering with the application) or at other times.

In certain embodiments, the system 100 and methods may provide an ability to assess, evaluate, and/or improve mental health of individuals without the need for explicit questions or the presence of a physician or therapist with the individuals. In operation, the system and methods may include capturing signals, content and/or data associated with an individual's mood and/or mental state from devices, applications, and/or systems that are utilized to interact with individuals. Additionally, in certain embodiments, other engagement conducted by individuals with an application, such as an individual's choice of content, participation in activities, completion of activities or content, along with the captured signals, content and/or data may be utilized by the system 100 and methods to assess and/or evaluate an individual's mental health and/or wellness. In certain embodiments, signals including any amount of the content and/or data may be labeled based on an assessment of how the individual's engagement with specific content should be interpreted based on a detailed framework determined using, for example, behavioral health experts.

The system 100 and methods may assign a score value (e.g., 0-100, 0-1, or other score within a range of values) to the labeled signals (and/or interactions) that assists in determining a specific mental health deficit or need that an individual might have that may be addressed by providing additional content or interactions with the applications, devices, and/or systems. As the individual conducts additional interactions with additional content on the applications, devices, and/or systems, the system 100 and methods may include dynamically recalculating the score value with each interaction, and the recalculated score value may be utilized to predict what further pieces of content, care activities, coaching, crisis support, therapy, and/or other potential mental health recommendations may be best suited to improve the individuals mental health state and overall well-being over time.

In certain embodiments, data and/or content associated with individuals and the individual's interactions with the system 100, applications, and/or devices may be loaded into artificial intelligence and/or machine learning models that have been trained to recognize patterns, behaviors, moods, feelings, actions, and/or other detectable characteristics associated with mental health. Such artificial intelligence models may be trained to recognize the patterns, behaviors objects, activities, individuals, and/or other items of interest based on analyzing other content and/or data that have been fed into the models on previous occasions. The effectiveness and detection capability of the artificial intelligence models may be enhanced as the models receive additional content and/or data over time, such as content and/or data resulting from further interactions with the individual or other individuals, such as individuals that may have a correlation with the mental health of the individual. The captured content and/or data may be compared to the content and/or data used to train the models and/or to deductions, reasoning, intelligence, correlations, outputs, analyses, and/or other information that the artificial intelligence model(s) learned based on the content and/or data used to train the models. The score values, mental health assessments and/or evaluations, and/or predictions may be generated using the artificial intelligence model(s) and machine learning. The labels, scores, assessments, evaluations, and/or mood improvement objective functions may be utilized to promote emotional and/or mental wellness.

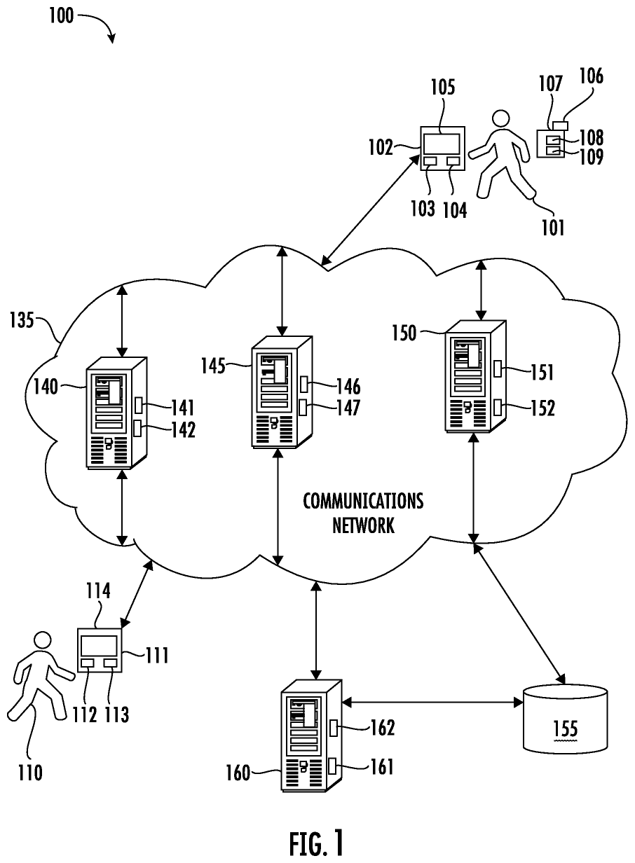

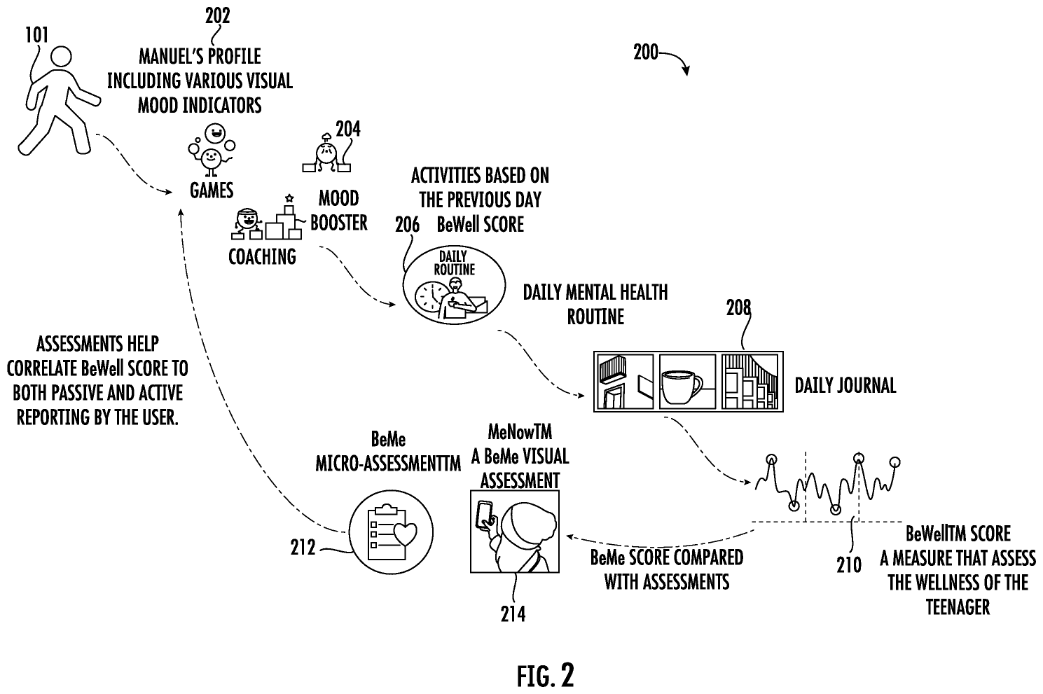

As shown in FIG. 1 and referring also to FIGS. 2-10, a system 100 and accompanying methods (e.g., method 900) for facilitating mental health assessment and enhancing mental health via facial recognition and content associated with physical attributes, motions, and expressions are disclosed. Notably, the system 100 may be configured to support, but is not limited to supporting, mental health systems and services, mental health improvement systems and services, monitoring systems and services, facial recognition systems and services, sensor devices and systems (e.g., sensors for measuring and detecting physical attributes, expressions, actions, etc. associated with a user), alert systems and services, data analytics systems and services, data collation and processing systems and services, artificial intelligence services and systems, machine learning services and systems, security systems and services, content delivery services, cloud computing services, satellite services, telephone services, voice-over-internet protocol services (VoIP), software as a service (SaaS) applications, platform as a service (PaaS) applications, gaming applications and services, social media applications and services, operations management applications and services, productivity applications and services, mobile applications and services, and/or any other computing applications and services. Notably, the system 100 may include a first user 101, who may utilize a first user device 102 to access data, content, and services, or to perform a variety of other tasks and functions. As an example, the first user 101 may utilize first user device 102 to transmit signals to access various online services and content, such as those available on an internet, on mobile devices, on other devices, and/or on various computing systems. As another example, the first user device 102 may be utilized to access an application, devices, and/or components of the system 100 that provide any or all of the operative functions of the system 100. In certain embodiments, the first user 101 may be any type of person, a robot, a humanoid, a program, a computer, any type of user, or a combination thereof. In certain embodiments, the first user 101 may be a person that may want to have their mental health assessed and/or evaluated, confirm whether a self-assessed emotional state is accurate, determine their emotional state based on physical attributes and/or expressions, seek assistance with improving their mental health, and/or seek to participate in activities associated with enhancing or maintaining mental health.

The first user device 102 may include a memory 103 that includes instructions, and a processor 104 that executes the instructions from the memory 103 to perform the various operations that are performed by the first user device 102. In certain embodiments, the processor 104 may be hardware, software, or a combination thereof. The first user device 102 may also include an interface 105 (e.g. screen, monitor, graphical user interface, etc.) that may enable the first user 101 to interact with various applications executing on the first user device 102 and to interact with the system 100. In certain embodiments, the first user device 102 may be and/or may include a computer, any type of sensor, a laptop, a set-top-box, a tablet device, a phablet, a server, a mobile device, a smartphone, a smart watch, and/or any other type of computing device. Illustratively, the first user device 102 is shown as a smartphone device in FIG. 1. In certain embodiments, the first user device 102 may be utilized by the first user 101 to control and/or provide some or all of the operative functionality of the system 100.

In addition to using first user device 102, the first user 101 may also utilize and/or have access to additional user devices. As with first user device 102, the first user 101 may utilize the additional user devices to transmit signals to access various online services and content. The additional user devices may include memories that include instructions, and processors that executes the instructions from the memories to perform the various operations that are performed by the additional user devices. In certain embodiments, the processors of the additional user devices may be hardware, software, or a combination thereof. The additional user devices may also include interfaces that may enable the first user 101 to interact with various applications executing on the additional user devices and to interact with the system 100. In certain embodiments, the first user device 102 and/or the additional user devices may be and/or may include a computer, any type of sensor, a laptop, a set-top-box, a tablet device, a phablet, a server, a mobile device, a smartphone, a smart watch, and/or any other type of computing device, and/or any combination thereof. Sensors may include, but are not limited to, cameras, wearable devices (e.g., wearable devices, digital wristbands, etc.), motion sensors, facial-recognition sensors, acoustic and audio sensors, pressure sensors, temperature sensors, light sensors, heart-rate sensors, blood pressure sensors, sweat detection sensors, breath-detection sensors, stress-detection sensors, blood glucose sensors, any type of health sensor, humidity sensors, any type of sensors, or a combination thereof.

The first user device 102 and/or additional user devices may belong to and/or form a communications network. In certain embodiments, the communications network may be a local, mesh, or other network that enables and/or facilitates various aspects of the functionality of the system 100. In certain embodiments, the communications network may be formed between the first user device 102 and additional user devices through the use of any type of wireless or other protocol and/or technology. For example, user devices may communicate with one another in the communications network by utilizing any protocol and/or wireless technology, satellite, fiber, or any combination thereof. Notably, the communications network may be configured to communicatively link with and/or communicate with any other network of the system 100 and/or outside the system 100.

In certain embodiments, the first user device 102 and additional user devices belonging to the communications network may share and exchange data with each other via the communications network. For example, the user devices may share information relating to the various components of the user devices, information associated with images and/or content accessed by a user of the user devices, information identifying the locations of the user devices, information indicating the types of sensors that are contained in and/or on the user devices, information identifying the applications being utilized on the user devices, information identifying how the user devices are being utilized by a user, information identifying user profiles for users of the user devices, information identifying device profiles for the user devices, information identifying the number of devices in the communications network, information identifying devices being added to or removed from the communications network, any other information, or any combination thereof. In certain embodiments, the user devices may share content obtained via sensors of the devices, such as, but not limited to, video content, audio content, haptic content, vibration content, augmented reality content, virtual reality content, sensor data (e.g., heart-beat data, blood pressure data, sweat data, respiratory data, breathing data, breath data, motion data (e.g., motion of limbs or other body parts), stress data, any other sensor data, or a combination thereof. In certain embodiments, the content obtained via the sensors may be associated with or of the first user 101 and may include measurements or information indicative of an emotional state of the first user 101, a mood of the first user 101, mental state of the first user 101, or a combination thereof. For example, such content may include facial expressions, body or body part movements, sweating, blood pressure drops or increases, glucose levels, body stiffness, speech rate, speech tone, speech volume, body position, any other physical expressions or attributes, or a combination thereof.

In addition to the first user 101, the system 100 may also include a second user 110. In certain embodiments, for example, the second user 110 may be another person that may seek to assess and/or evaluate her mental health, confirm self-assessments of mental health, moods, and/or emotional states, and improve upon her mental health and/or overall well-being. In certain embodiments, the second user 110 may be a mental health professional, such as, but not limited to, a psychiatrist, a therapist, a psychologist, and/or other mental health professional. In certain embodiments, the second user device 111 may be utilized by the second user 110 to transmit signals to request various types of content, services, and data provided by and/or accessible by communications network 135 or any other network in the system 100. In further embodiments, the second user 110 may be a robot, a computer, a vehicle, a humanoid, an animal, any type of user, or any combination thereof. The second user device 111 may include a memory 112 that includes instructions, and a processor 113 that executes the instructions from the memory 112 to perform the various operations that are performed by the second user device 111. In certain embodiments, the processor 113 may be hardware, software, or a combination thereof. The second user device 111 may also include an interface 114 (e.g. screen, monitor, graphical user interface, etc.) that may enable the first user 101 to interact with various applications executing on the second user device 111 and, in certain embodiments, to interact with the system 100. In certain embodiments, the second user device 111 may be a computer, a laptop, a set-top-box, a tablet device, a phablet, a server, a mobile device, a smartphone, a smart watch, and/or any other type of computing device. Illustratively, the second user device 111 is shown as a mobile device in FIG. 1. In certain embodiments, the second user device 111 may also include sensors, such as, but are not limited to, cameras, audio sensors, motion sensors, pressure sensors, temperature sensors, light sensors, heart-rate sensors, blood pressure sensors, sweat detection sensors, breath-detection sensors, stress-detection sensors, any type of health sensor, humidity sensors, any type of sensors, or a combination thereof.

In certain embodiments, the first user device 102, the additional user devices, and/or potentially the second user device 111 may have any number of software applications and/or application services stored and/or accessible thereon. For example, the first user device 102, the additional user devices, and/or potentially the second user device 111 may include applications for controlling and/or accessing the operative features and functionality of the system 100, applications for controlling and/or accessing any device of the system 100, interactive social media applications, biometric applications, cloud-based applications, VoIP applications, other types of phone-based applications, product-ordering applications, business applications, e-commerce applications, media streaming applications, content-based applications, media-editing applications, database applications, gaming applications, internet-based applications, browser applications, mobile applications, service-based applications, productivity applications, video applications, music applications, social media applications, any other type of applications, any types of application services, or a combination thereof. In certain embodiments, the software applications may support the functionality provided by the system 100 and methods described in the present disclosure. In certain embodiments, the software applications and services may include one or more graphical user interfaces so as to enable the first and/or potentially second users 101, 110 to readily interact with the software applications. The software applications and services may also be utilized by the first and/or potentially second users 101, 110 to interact with any device in the system 100, any network in the system 100, or any combination thereof. In certain embodiments, the first user device 102, the additional user devices, and/or potentially the second user device 111 may include associated telephone numbers, device identities, or any other identifiers to uniquely identify the first user device 102, the additional user devices, and/or the second user device 111.

In certain embodiments, the system 100 may optionally include any number or types of sensor devices 107. In certain embodiments, the sensor devices 107 may include a memory 108 that stores instructions and a processor 109 that is configured to execute the instructions to perform various operations of the sensor device 107. In certain embodiments, the memory 108 and processor 109 may be hardware, software, or a combination of hardware and software. In certain embodiments, the sensor device 107 does not need to include the memory 108 and/or processor 109. In certain embodiments, the sensor device may include communication devices 106 to facilitate transfer of data to and from the sensor device 107. In certain embodiments, the communication device 106 may include an antenna, a cellular communication module, a short-range wireless module (e.g., Bluetooth, etc.), a WiFi module, a radio frequency transmitter/reader, any type of communication device, or a combination thereof. In certain embodiments, the sensor device 107 may be worn by a user (e.g., first user 101, second user 110, and/or other users), in proximity to the user, on the user, in communication range of the first user device 102 and/or second user device 111, located in an environment, or a combination thereof. In certain embodiments, any number of sensor devices 107 may be utilized to generate and transmit sensor data to any of the components of the system 100 and/or outside of the system 100.

In certain embodiments, the sensor device 107 may be a camera configured to capture video content, audio content, audiovisual content, augmented reality content, content utilized for virtual reality content, motion content, or a combination thereof. For example, the camera may be configured to capture an image of the user, video of the user, speech or sounds made by the user, motion of the user, content of an environment in which the user is located, any other content, or a combination thereof. In certain embodiments, the sensor device 107 may be an audio sensor configured to capture sounds made by a user (including tone, pitch, volume, nervousness, anxiety, accent, etc.), sounds occurring in an environment in which a user is located, or a combination thereof. In certain embodiments, the sensor device 107 may be a motion sensor, which may be configured to capture motions conducted by the user (e.g., limb movement, body movement, head movements, eye movements, toe and finger movements, mouth movements, facial expressions, body expressions (e.g., body in a specific configuration or stance), speed of movements, angles of movements, types of movements, etc.). In certain embodiments, the sensor device 107 may be a pressure sensor, configured to detect pressure readings in an environment in which the user is located. In certain embodiments, the sensor device 107 may be a temperature sensor, which may be configured to detect the user's body temperature, a temperature of an environment, or a combination thereof.

In certain embodiments, the sensor device 107 may be a light sensor, which may be configured to measure light levels in an environment that the user is located in, the presence of the light, or a combination thereof. In certain embodiments, the sensor device 107 may be a heart-rate sensor, which may be configured to measure a heart rate of a user. In certain embodiments, the sensor device 107 may be a blood pressure sensor, which may be configured to measure blood pressure readings of the user. In certain embodiments, the sensor device 107 may be a sweat detection sensors, which may be configured to detect sweat perspired by a user. In certain embodiments, the sensor device 107 may be a breath-detection sensor, which may be configured to detect the rate at which a user is breathing, how deep a user is breathing, whether a user is breathing, smells in a user's breath, or a combination thereof. In certain embodiments, the sensor device 107 may be a stress-detection sensor, which may be configured to detect whether the user is stressed, such as by detecting stress hormones, excess breathing rate, tightening of muscles and the body, increases in volume of speech, changes in tone of speech, any other stress-related measurements, or a combination thereof. In certain embodiments, the sensor device 107 may be a vibration sensor configured to detect body of the user shaking or vibrating and/or vibrations occurring in an environment in which the user is located. In certain embodiments, the sensor device 107 may be any type of health sensor, humidity sensors (e.g., measures humidity in an environment and humidity in proximity to the user), any type of sensors, or a combination thereof.

The system 100 may also include a communications network 135. The communications network 135 may be under the control of a service provider, a business providing access to one or more applications supporting the functionality of the system 100, the first user 101, any other designated user, a computer, another network, or a combination thereof. The communications network 135 of the system 100 may be configured to link each of the devices in the system 100 to one another. For example, the communications network 135 may be utilized by the first user device 102 to connect with other devices within or outside communications network 135. Additionally, the communications network 135 may be configured to transmit, generate, and receive any information and data traversing the system 100. In certain embodiments, the communications network 135 may include any number of servers, databases, or other componentry. The communications network 135 may also include and be connected to a mesh network, a local network, a cloud-computing network, an IMS network, a VoIP network, a security network, a VoLTE network, a wireless network, an Ethernet network, a satellite network, a broadband network, a cellular network, a private network, a cable network, the Internet, an internet protocol network, MPLS network, a content distribution network, any network, or any combination thereof. Illustratively, servers 140, 145, and 150 are shown as being included within communications network 135. In certain embodiments, the communications network 135 may be part of a single autonomous system that is located in a particular geographic region or be part of multiple autonomous systems that span several geographic regions.

Notably, the functionality of the system 100 may be supported and executed by using any combination of the servers 140, 145, 150, and 160. The servers 140, 145, and 150 may reside in communications network 135, however, in certain embodiments, the servers 140, 145, 150 may reside outside communications network 135. The servers 140, 145, and 150 may provide and serve as a server service that performs the various operations and functions provided by the system 100. In certain embodiments, the server 140 may include a memory 141 that includes instructions, and a processor 142 that executes the instructions from the memory 141 to perform various operations that are performed by the server 140. The processor 142 may be hardware, software, or a combination thereof. Similarly, the server 145 may include a memory 146 that includes instructions, and a processor 147 that executes the instructions from the memory 146 to perform the various operations that are performed by the server 145. Furthermore, the server 150 may include a memory 151 that includes instructions, and a processor 152 that executes the instructions from the memory 151 to perform the various operations that are performed by the server 150. In certain embodiments, the servers 140, 145, 150, and 160 may be network servers, routers, gateways, switches, media distribution hubs, signal transfer points, service control points, service switching points, firewalls, routers, edge devices, nodes, computers, mobile devices, or any other suitable computing device, or any combination thereof. In certain embodiments, the servers 140, 145, 150 may be communicatively linked to the communications network 135, any network, any device in the system 100, or any combination thereof.

The database 155 of the system 100 may be utilized to store and relay information that traverses the system 100, cache content that traverses the system 100, store data about each of the devices in the system 100 and perform any other typical functions of a database. In certain embodiments, the database 155 may be connected to or reside within the communications network 135, any other network, or a combination thereof. In certain embodiments, the database 155 may serve as a central repository for any information associated with any of the devices and information associated with the system 100. Furthermore, the database 155 may include a processor and memory or may be connected to a processor and memory to perform the various operation associated with the database 155. In certain embodiments, the database 155 may be connected to the servers 140, 145, 150, 160, the first user device 102, the second user device 111, the sensor device 107, the additional user devices, any devices in the system 100, any process of the system 100, any program of the system 100, any other device, any network, or any combination thereof.

The database 155 may also store information and metadata obtained from the system 100, store metadata and other information associated with the first and second users 101, 110, store sensor data and/or content generated by the sensor 107, store features extracted from the sensor data and/or content, stores self-assessments made by a user, store journals made by a user (e.g., journals listing and tracking activities, behaviors, content experienced, and interactions by and/or with a user), store daily (or other time interval) mental health routines, store artificial intelligence algorithms supporting artificial intelligence models (and/or machine learning models) of the system 100 (e.g., algorithms supporting convolutional networks, vision transformers, recurrent neural networks, multiplayer perceptron networks, feed forward neural networks, long short-term memory networks, other artificial intelligence models and networks, or a combination thereof), store artificial intelligence models (and/or machine learning models) utilized in the system 100, store sensor data and/or content obtained from an environment associated with the first and/or second users 101, 110, store predictions made by the system 100 and/or artificial intelligence models (e.g., predictions for emotional states of uses based on analyzing content and/or sensor data associated with the user, predictions relating to which interactions, activities, and/or content are ideal for particular user, predictions relating to adjustments in scores for mental health based on types of interactions and/or activities that may be performed by a user, predictions relating to types of content that may be presented and/or delivered to a user to enhance mental health and/or scores, and/or any other predictions), store confidence scores relating to predictions made, store threshold values for confidence scores, store responses outputted and/or facilitated by the system 100, store information associated with anything detected, assessed, evaluated, and/or recommended via the system 100, store information and/or content utilized to train the artificial intelligence models, store information associated with behaviors and/or actions conducted by individuals with respect to the system 100, store user profiles associated with the first and second users 101, 110, store device profiles associated with any device in the system 100, store communications traversing the system 100, store user preferences, store information associated with any device or signal in the system 100, store information relating to patterns of usage relating to the user devices 102, 111, store any information obtained from any of the networks in the system 100, store historical data associated with the first and second users 101, 110, store device characteristics, store information relating to any devices associated with the first and second users 101, 110, store information associated with the communications network 135, store any information generated and/or processed by the system 100, store any of the information disclosed for any of the operations and functions disclosed for the system 100 herewith, store any information traversing the system 100, or any combination thereof. Furthermore, the database 155 may be configured to process queries sent to it by any device in the system 100.

Operatively, the system 100 may operate and/or execute the functionality as described in the methods (e.g. method 900 as described below) of the present disclosure. Additionally, the system 100 may incorporate the use of artificial intelligence models, machine learning models, and/or neural networks. In some embodiments, the exemplary inventive computer-based systems/platforms, the exemplary inventive computer-based devices, and/or the exemplary inventive computer-based components of the present disclosure may be configured to utilize one or more exemplary artificial intelligence/machine learning techniques chosen from, but not limited to, decision trees, boosting, support-vector machines, neural networks, nearest neighbor algorithms, Naive Bayes, bagging, random forests, and the like. In some embodiments and, optionally, in combination of any embodiment described above or below, an exemplary neutral network technique may be one of, without limitation, feedforward neural network, radial basis function network, recurrent neural network, convolutional network (e.g., U-net) or other suitable network. In some embodiments and, optionally, in combination of any embodiment described above or below, an exemplary implementation of Neural Network may be executed as follows: i) Define Neural Network architecture/model, ii) Transfer the input data to the exemplary neural network model, iii) Train the exemplary model incrementally, iv) determine the accuracy for a specific number of timesteps, v) apply the exemplary trained model to process the newly-received input data, vi) optionally and in parallel, continue to train the exemplary trained model with a predetermined periodicity.

In some embodiments and, optionally, in combination of any embodiment described above or below, the exemplary trained neural network model may specify a neural network by at least a neural network topology, a series of activation functions, and connection weights. For example, the topology of a neural network may include a configuration of nodes of the neural network and connections between such nodes. In some embodiments and, optionally, in combination of any embodiment described above or below, the exemplary trained neural network model may also be specified to include other parameters, including but not limited to, bias values/functions and/or aggregation functions. For example, an activation function of a node may be a step function, sine function, continuous or piecewise linear function, sigmoid function, hyperbolic tangent function, or other type of mathematical function that represents a threshold at which the node is activated. In some embodiments and, optionally, in combination of any embodiment described above or below, the exemplary aggregation function may be a mathematical function that combines (e.g., sum, product, etc.) input signals to the node. In some embodiments and, optionally, in combination of any embodiment described above or below, an output of the exemplary aggregation function may be used as input to the exemplary activation function. In some embodiments and, optionally, in combination of any embodiment described above or below, the bias may be a constant value or function that may be used by the aggregation function and/or the activation function to make the node more or less likely to be activated.

In certain embodiments, the system 100 may incorporate the use of convolutional neural networks to facilitate the determination and/or prediction of emotional states, mental states, or a combination thereof. In certain embodiments, the convolutional neural networks may be deep learning neural network tools that may be configured to process structured arrays (e.g., pixel arrays), such as images (or other types of content and/or sensor data) and may incorporate the use any number of convolutional layers that detect patterns in an input image (or other content). For example, such patterns may include, but are not limited to, lines, circles, gradients, faces, noses, smiles, frowns, wrinkles, skin tension, skin stretching, presence of sweat, presence of changes in skin color, presence of tears or changes in colors in eyes, and/or other patterns. In certain embodiments, each convolutional layer within the convolutional neural network can recognize more detailed and/or complex shapes and may be utilized to mirror the structure of a human visual cortex, which includes its own series of layers that process an image in front of an eye and identify increasingly complex features. In certain embodiments, each convolutional layer may include filters and/or kernels (e.g., matrices), which may be configured to slide over the input image (or other content) to determine patterns within the image that may correlate with patterns utilized to train the artificial intelligence/machine learning models of the system 100. If a certain part of the input image matches the pattern provided by the kernel, the kernel may return a large positive value, and, if the part does not match the pattern provided by the kernel, the kernel may return a zero or negative value. In certain embodiments, convolutional layers, for example, may include vertical line detectors, horizontal line detectors, diagonal detectors, corner detectors, curve detectors, among other detectors. Such detectors, for example, may be trained on image data and may be utilized to identify whether a particular thing (and subsequently a mood and/or emotional state) exists in an image. For example, the convolutional layers, using such detectors, can identify a smile within the image, which may indicate happiness or contentment.

In certain embodiments, the system 100 may incorporate the use of vision transformers to facilitate the predictions and/or detections. In certain embodiments, the vision transformers may be deep learning models that utilize mechanisms of attention, which differentially weight the significance of each part of the input data, such as an input image (or other content and/or sensor data). In certain embodiments, vision transformers may include multiple self-attention layers to facilitate computer vision-related tasks. In certain embodiments, a vision transformer may represent an input image (or other content and/or sensor data) as a series of image patches, flatten the image patches, generate lower-dimensional embeddings from the flattened image patches, provide positional embeddings, provide the embeddings as an input to a transformer encoder, pre-train the vision transformer model with image labels, and then fine-tine the dataset to perform a computer vision task, such as image classification, image segmentation, object or feature detection, content-based image retrieval or a combination thereof. In certain embodiments, a vision transformer encoder may identify local and global features that the image possesses. In certain embodiments, vision transformers may provide a higher precision rate on large datasets of images and/or other content, while also having reduced model training time.

Referring now also to FIG. 2, an exemplary process flow 200 for use with the system 100 for analyzing a user's profile, examining activities conducted by a user with an application of the system 100, determining a mental health routine for the user, compiling a daily journal of activities and/or interactions performed by the user, calculating the mental health score of the user, and comparing the score with user self-assessments and/or assessments made by the system 100 based on analyzing content associated with the user is shown. An exemplary use-case scenario for the process flow 200 may be as follows: The first user 101 (e.g., Manuel) may have a user profile 202 stored in the system 100. In certain embodiments, the user profile may include demographic information for the user, health information for the user, psychographic information for the user, mental health history for the user, vital information for the user, content associated with the user (e.g., images taken of the user, sensor data associated with the user, any other information, or a combination thereof), any other information, or a combination thereof. In certain embodiments, the user profile may include mood indicators associated with physical attributes of the user, expressions of the user, or a combination thereof. For example, the user profile may include mood indicators corresponding to facial features of the user. A mood indicator may indicate, for example, that when the user is frowning that the user is in a sad and/or depressed emotional state. Another mood indicator may indicate that when one side of the user's mouth is turn up and the other side is neither up nor down that the user is in a pondering emotional state. In certain embodiments, the system 100 may include, in the user profile, science and studies indicating the use of physical attributes (e.g. facial features and/or expressions) and motion cues (including muscle use) as being tied to specific mood indicators. In certain embodiments, the user profile may include indications as to what features of the user correlate to what emotional states.

In certain embodiments, the user profile may include information associated with activities 204 that the user is to perform, has performed, or a combination thereof. For example, the application supporting the system 100 may indicate that the user should read a book for 30 minutes and then run outside for 30 minutes. The user profile may indicate the user's wellness score before performance of the activities and the user's wellness score after the performance of the activities. The user profile may also include daily (or other time interval) mental health routines 206 for the user to perform. In certain embodiments, the user profile may also include a daily journal 208 that logs all the activities and content experienced by the user each day (or other time interval). In certain embodiments, the user profile may also include a score 208 (e.g., wellness score or other score described herein and/or incorporated herein, such as those described in U.S. Provisional Application No. 63/326,646) and accompanying graphs that show changes in the score over time. In certain embodiments, the score may be a measure that assesses the wellness of the user.

In certain embodiments, the system 100 may be configured to store all micro-assessments 212 in the system 100, such as in database 155. In certain embodiments, the micro-assessments may be self-assessments made by the user that indicate the user's emotional state as expressed in content taken of the user and/or sensor data associated with the user. In certain embodiments, the score 208 may be compared to the micro-assessments 212 to determine any deviations or inconsistencies. In certain embodiments, the user profile may also include predicted emotional state assessments 214 (e.g., emotional states predicted from content taken of and/or associated with the user). The score for the user may be compared to the micro-assessments 212 and/or predicted emotional state assessments 214 to determine deviations and/or whether there is alignment between the assessments and score. In certain embodiments, for example, the score for the user (e.g., as determined in U.S. Provisional Application No. 63/326,646 and/or as described in the present disclosure), which relates to the user's emotional state may be compared to the self-assessed emotional state provided by the user and any deviation from the score for the user and the self-assessed emotional state may be identified based on analyzing the physical attributes, expressions, or a combination thereof, detected in the content and/or sensor data associated with the user. In certain embodiments, the score 210 and/or assessments 212, 214 may be utilized to modify the score 210 and/or assessments 212, 214 accordingly. The user profile may also keep logs of how the user's emotional state changes over time and in response to performance of activities, non-performance of activities, experiencing content, not experiencing content, or a combination thereof.

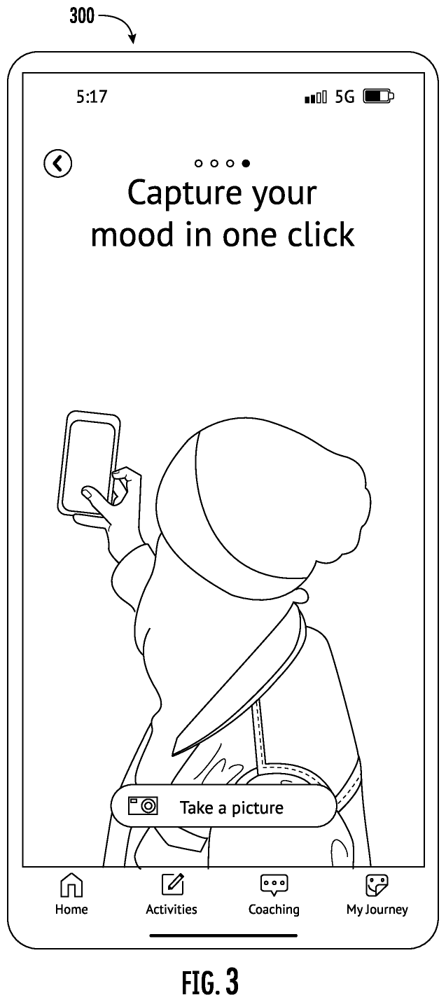

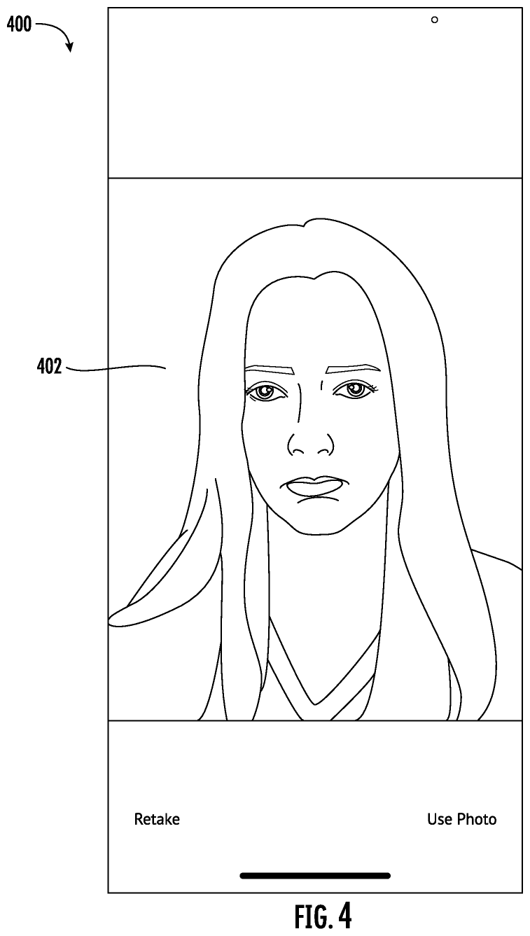

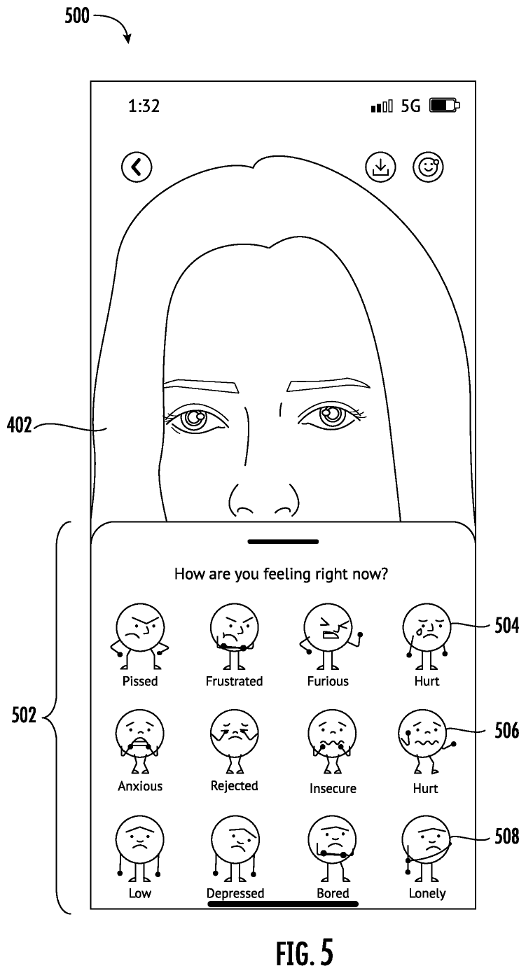

In certain embodiments, the system 100 may operate in the following exemplary use-case scenario. Referring now also to FIGS. 3, 4, 5, 6, 7, and 8, such a use-case scenario is shown. FIG. 3 illustrates an exemplary user interface 300 that provide the ability for a user to take a picture(s) (and/or other content, such as videos, audio, etc.) of herself to capture the user's mood using the camera of the first user device 102. User interface screen 400 of FIG. 4 illustrates an exemplary image 402 that the user took of herself in the application. In certain embodiments, the application enables the user to retake the image, use the image 402, and/or take additional images (or other content). Once the image 402 is taken, the application supporting the functionality of the system 100 may enable the user to tag the user's image with one or more self-assessed emotional states 502, which may include categories of emotion states, such as, but not limited to, angry emotional states 504 (row 1), anxious or insecure emotional states 506 (row 2), and sad emotional states 508 (row 3). The user interface 500 of FIG. 5 shows an exemplary self-assessed emotional states 502 including emojis that correlate with specific emotional states that the user may self-assess for the image 402 taken of the user. In certain embodiments, the self-assessed emotional states do not have to be associated with the image 402 taken of the user, but instead, could be an emotional state before or after taking the image 402.

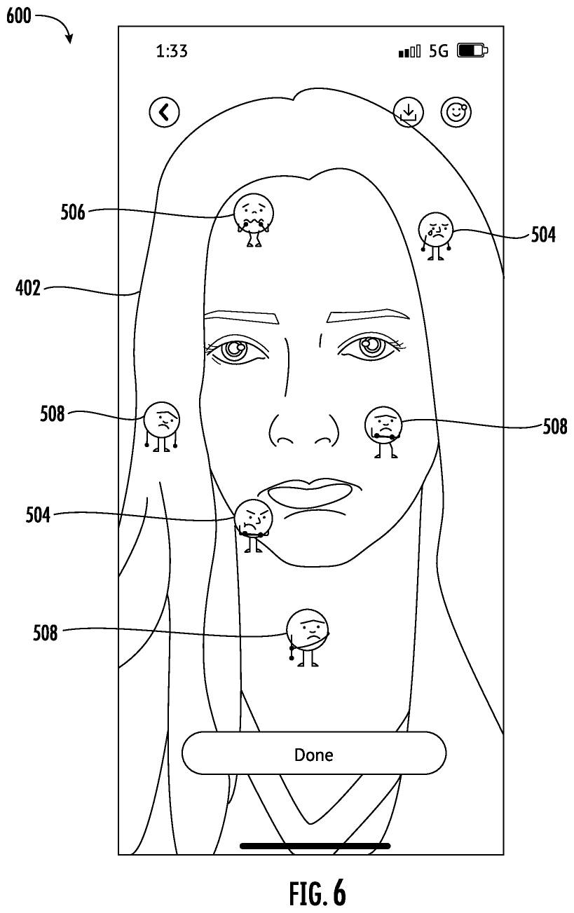

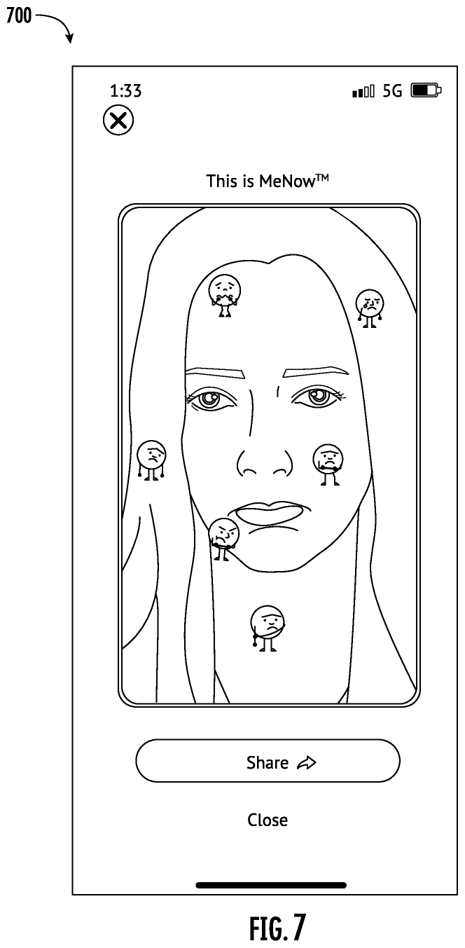

Referring now also to FIG. 6, an exemplary user interface 600 is shown, which visually depicts the tagged self-assessed emotional states on the image 402 taken of the user. For example, the user may have selected multiple emotional states within the sad emotional state 508 row, a single emotional state 506 from the anxious or emotional states row, and two emotional states from the angry emotional states 504 row. In certain embodiments, the selected self-assessed emotional states may be visually rendered via emojis and/or words on the image 402 itself for easy viewing. Once the self-assessed emotional states are selected and confirmed, the user may set the emotional states to the image 402. Referring now also to FIG. 7, an exemplary user interface 700 depicts the image 402 taken of the user with the self-assessed emotional states as a representation of the user at the current time (i.e., the user's “MeNow” state). In certain embodiments, the application may enable the user to share the user's MeNow images and state with other users, the application, and/or other devices and systems.

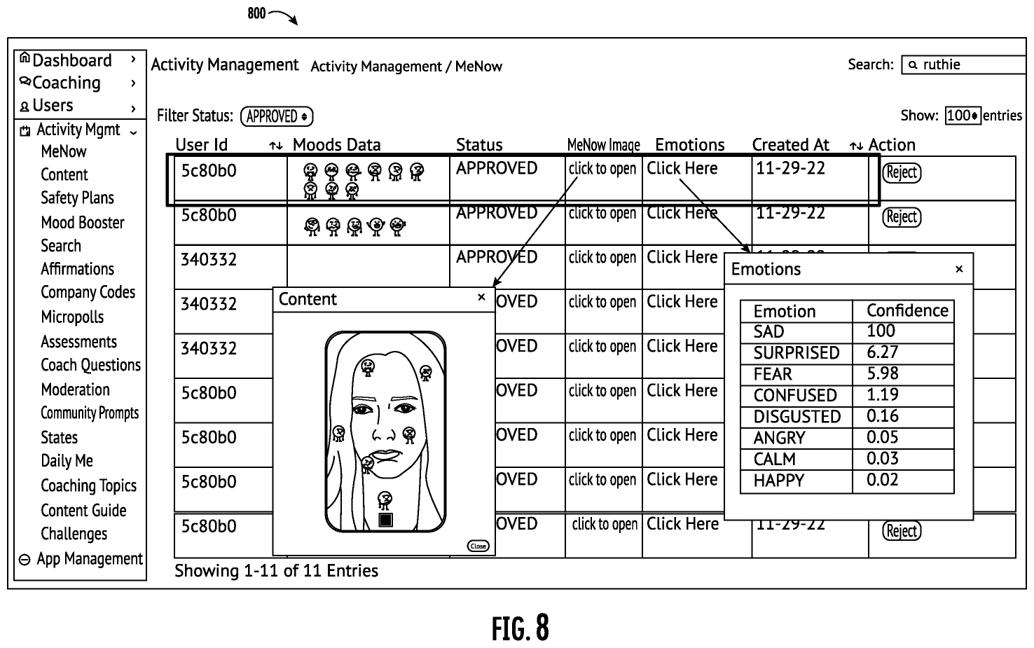

Referring now also to FIG. 8, an exemplary user interface 800 is shown, which visualizes further functionality of the system 100. In certain embodiments, the user interface 800 may include a plurality of rows and columns including various information for various users. For example, the first column can be a user identifier that identifies each unique user of the system 100, the second column may be moods data for each user, which may include self-assessed emotional states and/or predicted emotional states and which may be visualized via emojis and/or text, a third column may be an approval status of the user (e.g., approved or not approved), the fourth column may be a link to the provided MeNow image (or other content), the fifth column may be a list of emotions that the system 100 has determined and/or predicted for the user and corresponding confidence scores (e.g. expressed in percentages, from 1-100, 0-1, and/or any other manner) for each emotional state that the system 100 determined and/or identified for the user, the sixth column may indicate the date at which the predictions, assessments, and/or images were taken, and the seventh column indicates potential actions that the user should perform and/or content that the user should experience to enhance or maintain mental health, emotional health, or a combination thereof. In certain embodiments, the confidence scores may be determined based on a level of correlation with the content (e.g., image of the user) and/or sensor data with the emotion as determined based on comparison with training information utilized to train the machine learning and/or artificial intelligence models that indicate associations between emotions and physical attributes and/or expressions that may be correlated with physical attributes and/or expressions detected in the content and/or sensor data.

Notably, in certain embodiments, the functionality provided by the system 100 may be amplified by factoring in various types of markers (which may be included in the user profile) when predicting and/or identifying the user's emotional state, mental health state, or a combination thereof. For example, in certain embodiments, the markers such as, but not limited to, driving history, substance use, sexual risk behaviors, adherence to medication regimen, threat-avoidance, reward-pursuit/reward-seeking, risk-taking (including risk on health), diet and physical activity, greater education, goal setting, self-monitoring and parental involvement, new experiences (particularly related to social interaction), intimacy, romantic love, jealousy, targeted rejection acceptance, heightened impulsivity, sensation-seeking, reward sensitivity, seeking new experiences and social interactions, establishing identity, developing routines in new settings, sociocultural-political environment, context of user's peer, family, school, and neighborhood, generalized pessimism, dispositional optimism, other markers, or a combination thereof. Additional markers may include, but are not limited to, location context (e.g., urban/rural locations, school district, physical setting, etc.), socioeconomic status (which can effect stress levels, affecting overall mental health), physical attributes (e.g., height, weight, age, gender, eye color, hair color, etc.), racial/ethnic context (e.g., indicated or predicted), history of major or other surgeries, drug addiction and/or suicide attempts, life events (e.g., explicit knowledge or contextual such as holidays, travel, high stress periods like exams or major local or global events), emotional actions (e.g., attempted suicide, substance use, eating disorders, isolation, etc.), changes in facial features/facial coding (e.g., Head movement-looking down/away from camera, not being able to sit still, etc.; lips—a slight smirk can show joy, while pressed or lips may mean the user is anxious; eyebrows—furrowed brow could mean anger or frustration; wrinkle on forehead; eye twitch (may be a result of fatigue or stress); clenched jaw may signify anger; raising and lowering of mouth corners may signify happiness; lowering of mouth corners, raise inner portion of brows may signify sadness; eyebrows arch, eyes open wide to expose more white, jaw dropping slightly may signify surprise; eyebrows raised, eyes open, mouth opens slightly may signify afraid; upper lip is raised, nose bridge is wrinkled, cheeks raised may signify disgust; and brows lowered, lips pressed firmly, eyes bulging may signify anger), audio-related markers (e.g., voice cracks/markers/vocal cues, hesitations or speed of speech), major life events or daily hassles (across school, personal relations, peer relations and social/family conditions and may include, but are not limited to, loss/separation/grief, financial pressures, academic performance, school attendance, teacher interactions, family living conditions, romantic relationships, peer pressure, uncertainty regarding the future, conflict within school/leisure environment, and emerging adult responsibility), and time-related markers (e.g., use of past tense may signify depression, use of future tense may signify anxious or optimistic/hopeful, and use of present tense may signify being grounded/centered).

In certain embodiments, the system 100 may request users to explicitly capture various moods when they start using the application (e.g., at registration or at login). In certain embodiments, the system 100 may offer the users various activities including a feature named MeNow (as described above). In certain embodiments, the application may enable users to capture selfies and videos, along with various emotional tags or emojis at a particular instant or period of time. In certain embodiments, facial expressions may be both user provided and passively observed from the images across various activities including MeNow. In certain embodiments, mood images (or other content) may be captured at the application sign up by the user. In certain embodiments, pictures, videos, and/or other content and/or sensor data all may be used as features and/or vectors to facilitate predictions and confirmations of self-assessments by the system 100. In certain embodiments, such vectors could vary over time for the same user. In certain embodiments, the artificial intelligence/machine learning models supporting the system 100 may be built to predict the current state of emotion based on vectors collected over a time period until the current moment. In certain embodiments, the system 100 process above may return an outcome which may be the emotional state for the user at the moment. In certain embodiments, a score (e.g. wellness score of U.S. Provisional Application No. 63/326,646 and/or scores described herein) for the user may provide an indication of the user's emotional state as well. In certain embodiments, the system 100 may overlay both of these outcomes and convolute them to measure any delta or deviation between the two outcomes. In certain embodiments, the artificial intelligence/machine learning model may be continually trained to understand the delta/deviation and its meaning as related to the specific user. In certain embodiments, a clinical assessment score can also be used to compare against the detected emotions from MeNow and learn about the differences. In certain embodiments, training over time by the machine may assist in developing a statistical model (and even auto generate a program to generate the statistical model) that is able to generate an outcome—emotional state of a user, such as a teenager. In certain embodiments, the system 100 may assist in predicting the emotional state closer than any statistical assessment used in the current mental health industry.

In certain embodiments, the system 100 may enable users to perform activities that are categorized both on wellness categories and clinical categories. Based on the users' actions, the next set of actions may be offered, which in turn assist in improving the wellness of the user. The offered activities may be generated based on the functionality described herein and/or also algorithms (i.e., BeWell Algorithms) as described in U.S. Provisional Application No. 63/326,646, filed on Apr. 1, 2022. In certain embodiments, the system 100 utilizes machine learning models that may use the user provided images (or other content) to detect possible emotions and correlate them to the signals from the BeWell Algorithm to result in a more accurate prediction of the emotional state of the user.

In certain embodiments, the system 100 may enable users to take selfies or selfie videos and select any number of emotions to describe how they feel. For example, the users may be prompted to take images or video (or other content) of the user while the user is sad, happy, delighted, motivated, angry, scared, surprise, content, lonely, disgusted, agitated, relaxed, overwhelmed, tired, grieving, anxious, chill, low, upset, pumped, bored, creative, depressed, edgy, fearful, hungry, grateful, isolated, joyous, laughing, and/or other emotional and/or mental states. In certain embodiments, when a user takes a picture or selfie video, the user may input several emotions that express how they feel. Upon downloading the application supporting the system 100, the user may be asked to repeat a few phrases for the application to know what the user's voice sounds like. Later on, the user may be allowed to submit video/audio recordings of themselves into the application. Using the original recordings and audio markers, the system 100 may assess what the users are feeling through vocal cues. In certain embodiments, the artificial intelligence/machine learning model(s) scans the selfie or video to search for more accurate emotions that the user may be feeling. Users often do not know how to express their emotions or tell the system 100 application exactly how they feel. A facial scanning software may be utilized in providing a better idea about what a user is feeling. In certain embodiments, combining the data from the models and the information that the user inputs, the system 100 may assess the user's needs, predict what content would be most helpful, and deliver a series of recommendations that can be tracked within the application specific to a person's needs based on the individual pattern of prior behavior and also relative to other users who have expressed similar emotions and what activities have been deemed most useful by them. In certain embodiments, the system may allow users to reflect on their emotions over time. If they choose, users can see the log of emotions that they were feeling and reflect on their progress/changes, if any. In certain embodiments, the curated content given to users can be either generated by the application for the user (e.g., a notification to go on a walk) or the application could send content that is already part of the application (e.g., a video on breathing exercises).

Additionally, the artificial intelligence/machine learning models may be utilized to detect the users' emotions. Such models may be used to assess a user's feelings more accurately, because the user might not be completely honest. In certain embodiments, the emotion detection may be compared to emotions explicitly selected by users, so there is a comparison between the implicit and the explicit. In certain embodiments, in addition to the comparison with selected moods, the implicit mood detection can be matched to patterns of behavior in other parts of the application. In certain embodiments, because users may be encouraged to record their mood frequently, analysis of implicit mood changes can be compared to user mood awareness at various times throughout the day. In certain embodiments, differences in detected moods and indicated moods can help improve the quality of implicit mood detection through continuous label entry by users. In certain embodiments, the outcome of the mood detection and comparison can lead to recommended content, activities, professional medical support or crisis support that a system that only uses facial detection may not be able to accurately predict. In certain embodiments, the artificial intelligence/machine learning models may enable the application supporting the system 100 to provide a more accurate idea of how the users are feeling, and the application may utilize that information to give the users' videos and tools to deal with whatever emotions they may be feeling. In certain embodiments, the application may utilize the information about the user's mood to give the users content and activities that are specific to their needs, such as but not limited to, videos, events to attend, activities to participate in (go for a walk, draw something, talk to a friend, etc), appointments to make with a therapist, and coaching sessions to set. In certain embodiments, the system 100 may use the information gathered from the models and evaluate long-term mood changes to assess each user more accurately. Using a combination of artificial intelligence software and letting users choose from a list of emotions they may be feeling allows the system 100 to evaluate whether the user is being honest/aware about their moods.

Notably, as shown in FIG. 1, the system 100 may perform any of the operative functions disclosed herein by utilizing the processing capabilities of server 160, the storage capacity of the database 155, or any other component of the system 100 to perform the operative functions disclosed herein. The server 160 may include one or more processors 162 that may be configured to process any of the various functions of the system 100. The processors 162 may be software, hardware, or a combination of hardware and software. Additionally, the server 160 may also include a memory 161, which stores instructions that the processors 162 may execute to perform various operations of the system 100. For example, the server 160 may assist in processing loads handled by the various devices in the system 100, such as, but not limited to, receiving content associated with one or more physical attributes of a user, one or more expressions of the user, or a combination thereof, from one or more sensors; receiving one or more self-assessed emotional states being experienced by the user; extracting features from the content, such as by utilizing various artificial intelligence and neural network techniques; determining one or more predicted emotional states of the user by comparing the extracted features to information utilized to train artificial intelligence models facilitating in the determination; determining whether there is any deviation between the one or more self-assessments and the one or more predicted emotional states; generating and/or identifying content to deliver to the user and activities to recommend to the user to enhance or maintain the user's emotional state, mental state, or a combination thereof; providing the content and recommendations to the user; monitoring the user's emotional state and/or mental state during and/or after experiencing the content and/or participating in the activities; training the artificial intelligence models (and/or machine learning models) to enhance future predictions; and performing any other operations conducted in the system 100 or otherwise. In one embodiment, multiple servers 160 may be utilized to process the functions of the system 100. The server 160 and other devices in the system 100, may utilize the database 155 for storing data about the devices in the system 100 or any other information that is associated with the system 100. In one embodiment, multiple databases 155 may be utilized to store data in the system 100.

Although FIGS. 1-10 illustrates specific example configurations of the various components of the system 100, the system 100 may include any configuration of the components, which may include using a greater or lesser number of the components. For example, the system 100 is illustratively shown as including a first user device 102, a second user device 111, sensor device 107, a communications network 135, a server 140, a server 145, a server 150, a server 160, and a database 155. However, the system 100 may include multiple first user devices 102, multiple second user devices 111, multiple sensor devices 107, multiple communications networks 135, multiple servers 140, multiple servers 145, multiple servers 150, multiple servers 160, multiple databases 155, or any number of any of the other components inside or outside the system 100. Furthermore, in certain embodiments, substantial portions of the functionality and operations of the system 100 may be performed by other networks and systems that may be connected to system 100.

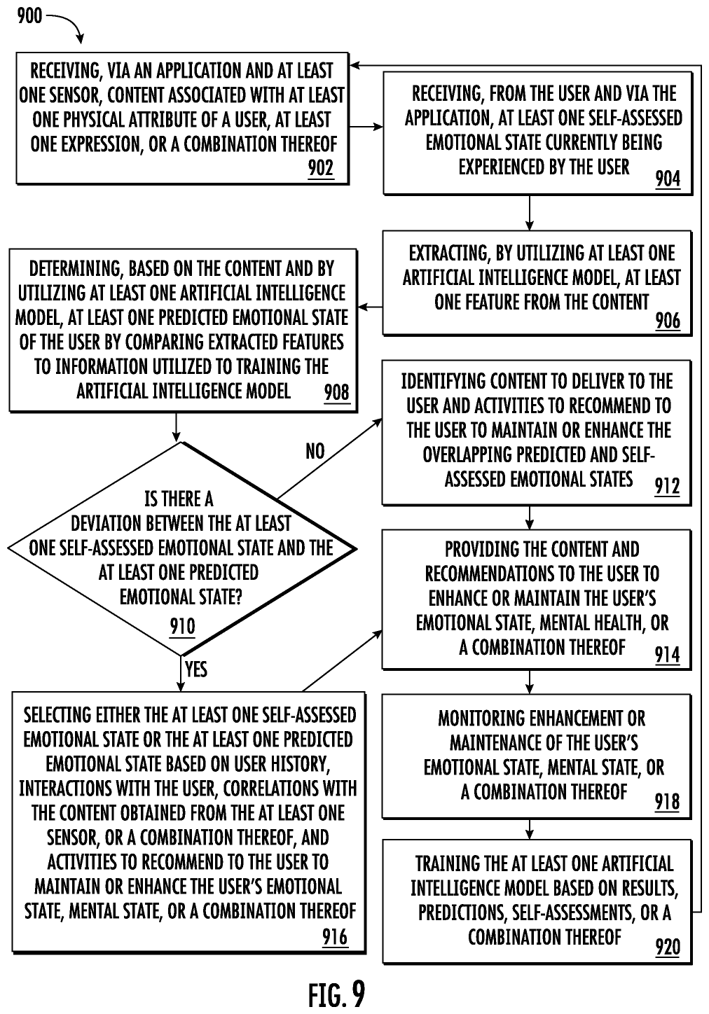

Notably, the system 100 may execute and/or conduct the functionality as described in the exemplary method(s) that follow. As shown in FIG. 9, an exemplary method 900 for facilitating mental health assessment and enhancing mental health via facial recognition and content associated with physical attributes, expressions, or a combination thereof, is schematically illustrated. The method 900 and/or functionality and features supporting the method 900 may be conducted via an application of the system 100, devices of the system 100, processes of the system 100, any component of the system 100, or a combination thereof. The method 900 may include steps for receiving content associated with physical attributes, expressions, and/or other characteristics associated with a user, such as from one or more sensors (e.g., sensor device 107), receiving self-assessed emotional states from the user, extracting features from the content, determining predicted emotional states for the user based on comparing the features to information utilized to train artificial intelligence models of the system 100, identifying content to deliver to the user to enhance or maintain the user's emotional and/or mental state, generating recommendations for activities for the user to perform to enhance or maintain the user's emotional and/or mental state, providing the content and/or recommendations to the user, tracking the user's progress, and training the artificial intelligence models supporting the functionality provided by the system 100, the method 900, or a combination thereof.

At step 902, the method 900 may include receiving, via an application, content associated with at least one physical attribute of a user, at least one expression, or a combination thereof. In certain embodiments, the physical attributes may be any type of physical attribute of the user, such as, but not limited to, skin color, hair type, hair color, height, weight, birthmarks, sweat, eyebrows, nose, limbs, tears, eye color, facial and/or body shape, body dimensions, heart rate, breathing rate, body temperature, any type of sensor data, any physical attribute, or a combination thereof. In certain embodiments, expressions may be facial expressions (e.g., smile, frown, angry face, blinking, etc.), body configurations, stances, any other types of expressions, or a combination thereof. In certain embodiments, for example, the content may be obtained by utilizing sensor devices 107, the first user device 102 (e.g. sensors of the first user device 102), the second user device 111, any other devices, or a combination thereof. In certain embodiments, the content may be video content and/or audio content captured by a camera (e.g., sensor device 107). In certain embodiments, the content may be image content, such as a digital photo taken by the first user device 102 and/or by a camera (e.g., sensor device 107). For example, the image content could be an image of the first user's 101 face containing the current facial expression of the first user 101 at a particular moment in time. In certain embodiments, measurements, such as, but not limited to, the first user's 101 heart rate, blood pressure, sweat levels, body movements, breathing rate or depth, body tension, any other measurements, or a combination thereof. In certain embodiments, the receiving of the content may be performed and/or facilitated by utilizing the first user 101, the second user 110 and/or by utilizing the first user device 102, the second user device 111, the sensor device 107, the server 140, the server 145, the server 150, the server 160, the communications network 135, any component of the system 100, any combination thereof, or by utilizing any other appropriate program, network, system, or device.

At step 904, the method 900 may include receiving one or more self-assessed emotional states currently being experienced by the user. In certain embodiments, the self-assessed emotional states may be the emotions that the user is experiencing in the content received from the one or more sensors (e.g., what emotions the user is experiencing in an image taken of the user by a camera of the first user device 102), emotions that the user is experiencing in general, emotions that the user is experiencing during a particular period of time, or a combination thereof. In certain embodiments, the self-assessed emotional states may be what the user considers her own personal emotional states to be and are designated by herself. For example, the self-assessed emotional states may indicate that the user is happy, sad, angry, frustrated, anxious, nervous, irritated, depressed, furious, hurt, rejected, insecure, bored, lonely, In certain embodiments, receiving of the one or more self-assessed emotional states may be performed and/or facilitated by utilizing the first user 101, the second user 110 and/or by utilizing the first user device 102, the second user device 111, the sensor device 107, the server 140, the server 145, the server 150, the server 160, the communications network 135, any component of the system 100, any combination thereof, or by utilizing any other appropriate program, network, system, or device.

At step 906, the method 900 may include extracting, by utilizing one or more artificial intelligence models (and/or machine learning models), one or more features from the content. For example, in certain embodiments, convolutional neural networks and layers of a neural network associated with the artificial intelligence models may be utilized to extract features from the content (e.g., an image of the user). In certain embodiments, the artificial intelligence models may be configured to generate a feature map for the content, which may be utilized to divide the content into media content patches (e.g., image or other types of content patches) of a selected or random size that may be converted into vectors that may be processed by the neural network. In certain embodiments, the vectors may comprise numbers representing pixels (or other content units) of the content that may be fed into a machine learning classification, segmentation, detection, or other artificial intelligence system for processing. In certain embodiments, the extracting of the features may be performed and/or facilitated by utilizing the first user device 102, the second user device 111, the server 140, the server 145, the server 150, the server 160, the communications network 135, any component of the system 100, any combination thereof, or by utilizing any other appropriate program, network, system, or device.

At step 908, the method 900 may include determining, based on the features of the content and by utilizing one or more artificial intelligence models, one or more predicted emotional states of the user. In certain embodiments, the determination of the predicted emotional state may by conducted by comparing the extracted features to information utilized to train the artificial intelligence models. For example, information utilized to train the artificial intelligence models may indicate that certain patterns (e.g., a frown, smile, furrowed eyebrows, squinted eyes, puckered mouth, etc.) in images are associated with certain emotional states. If one or more features of the content have a threshold level of correlation or match the patterns associated with certain emotional states utilized to train the artificial intelligence model, the system 100 may predict that the emotional state of the user corresponds to the certain emotional states utilized to train the artificial intelligence model. For example, if the features in the content indicate that the user is frowning and the user's lips are turned downwards, the system's 100 artificial intelligence models may have been trained with images of frowning individuals that have been tagged as being associated with sadness, depression, and/or other associated emotional states. In such a scenario, the artificial intelligence models (and/or machine learning models) of the system 100 may predict that the emotional state of the user is that the user is sad and depressed. Similarly, for other types of content, such as audio content of the user (e.g., user's speech), the system 100 may predict the emotional state based on comparing patterns, tones, volumes, pitches, intensity, types of words being used, etc. in the content and/or sensor data that are associated with the user to audio information utilized to train the models that is tagged or marked as being correlated with and/or associated with certain emotional and/or mental states. For example, a specific pattern in the user's speech may correlate with a pattern known to a machine learning model as being associated with insecurity or nervousness. Still further, for sensor data patterns in the sensor data may be compared to sensor data utilized to train the models to identify emotional states based on the sensor data (e.g., high heart rate may indicate anxious emotional state). As another example, tension in a user's neck may be determined by the system 100 to be associated with nervousness, anger, stress, or a combination thereof. In certain embodiments, the determining of the one or more predicted motional states may be performed and/or facilitated by utilizing the first user 101, the second user 110 and/or by utilizing the first user device 102, the second user device 111, the sensor device 107, the server 140, the server 145, the server 150, the server 160, the communications network 135, any component of the system 100, any combination thereof, or by utilizing any other appropriate program, network, system, or device.

At step 910, the method 900 may include determining whether there is a deviation between the one or more self-assessed emotional states provided by the user and the one or more predicted emotional states predicted by the artificial intelligence models of the system 100. For example, the system 100 may determine that the user self-assessed herself as being sad and depressed and tagged an image taken of the user as being sad and depressed. The artificial intelligence models may separately analyze the image and extract features from the image. The features may be compared to information relating to emotional states that was utilized to train the artificial intelligence models. In certain embodiments, the artificial intelligence models may determine that the features extracted from the content have a threshold correlation with sadness and depression. In such a scenario, there may be no deviation or threshold deviation between the predicted emotional states and the self-assessed emotional states. In certain embodiments, the determining of whether a deviation exists may be performed and/or facilitated by utilizing the first user 101, the second user 110 and/or by utilizing the first user device 102, the second user device 111, the sensor device 107, the server 140, the server 145, the server 150, the server 160, the communications network 135, any component of the system 100, any combination thereof, or by utilizing any other appropriate program, network, system, or device.

In the event that there is no deviation or threshold deviation, the method 900 may proceed to step 912. At step 912, the method 900 may include identifying content (or resources) to deliver to the user and activities to recommend to the user to maintain or enhance the overlapping predicted and self-assessed emotional states. For example, the system 100, such as by utilizing the artificial intelligence models, may determine that the user should walk for thirty minutes and also watch a 5-minute video of a person walking through a field with rabbits, deer, flowers, and other positive imagery to boost the user's mental health, emotional, and even physical health. In certain embodiments, at step 912, the method 900 may include generating the content to be delivered to the user to maintain or enhanced the user's emotional and/or mental state. In certain embodiments, the identifying, the recommending, and/or the generating of the content and activities may be performed and/or facilitated by utilizing the first user 101, the second user 110 and/or by utilizing the first user device 102, the second user device 111, the sensor device 107, the server 140, the server 145, the server 150, the server 160, the communications network 135, any component of the system 100, any combination thereof, or by utilizing any other appropriate program, network, system, or device.

In certain embodiments, the content, activities, and/or resources may include, but are not limited to, surveys, gaming content, mood booster content, videos, audio content, augmented reality content, virtual reality content, therapy sessions, communication sessions, appointments with friends, training sessions, virtual meetings with mental health experts, interactive content, virtual reality content, augmented reality content, quizzes, data extraction programs, questionnaires, activities, physical exercise, breathing exercise programs, meditation programs, and/or other types of content, activities, and/or resources. In certain embodiments, the system 100 may identify content to deliver to the user based on the content being pre-tagged in the system 100 or by other systems as being effective with enhancing a maintaining a particular type of mood. In certain embodiments, the system 100 may attempt to do a trial and error approach and select content and/or activities based on a predicted probability of success (e.g., content worked on other users and/or is likely to enhance or maintain mood based on the type of content contained therein) and track how the user's emotional state changes after the user experiences such content and/or activities.

At step 914, the method 900 may include providing the content and/or recommendations to the user to facilitate enhancement or maintenance of the user's emotional state, mental state, other states (e.g., physical, meditative, restful, etc.), or a combination thereof. In certain embodiments, the providing of the content and/or recommendations may be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device. If, however, at step 910, there is a deviation or threshold deviation between the one or more self-assessed emotional states and the one or more predicted emotional states, the method 900 may proceed to step 916. A deviation may exist if the predicted emotional state is different from the self-assessed emotional state, if there is a certain number of different characteristics (e.g., happiness may have characteristics that include a smile, steady heart rate, no perspiration, slightly squinted eyes, being in a happy location, etc.) between the predicted emotional state and the self-assessed emotional state, if the predicted emotional state and the self-assessed emotional state do not have a threshold number of overlapping characteristics, if the correlation with the content of the predicted emotional state and the self-assessed emotional state is greater than a threshold amount (e.g., percentage), or a combination thereof.

At step 916, the method 900 may include selecting either the one or more self-assessed emotional state of the one or more predicted emotional states for the user. In certain embodiments, the selection of either the self-assessed emotional state or the predicted emotional state may be based on the user's history of interactions with the application supporting the system 100, based on the user's activities, based on the user's mood history over time, based on the user's diagnoses from a mental health professional, based on the content correlating more (e.g., higher percentage) with the predicted or the self-assessed emotional state, based on a history of the user not being truthful, based on other aspects, or a combination thereof. In certain embodiments, the selecting may be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device. In certain embodiments, the selection may even be of another emotional state that is different from the self-assessed emotional state or the predicted emotional state. For example, the selection may be of an emotional state that has characteristics of both the predicted emotional and the self-assessed emotional state but is different than both. In certain embodiments, the system 100 may select a combination of the predicted emotional state and the self-assessed emotional state.

Once the selection is made at step 916, the method 900 may proceed to step 914, which may include providing content and/or recommendations to the user to enhance or maintain the user's emotional state, mental health, or a combination thereof. In certain embodiments, the content may be provided to the user's device (e.g., first user device 102, second user device 111, and/or another device), to another device or system, or a combination thereof. In certain embodiments, the providing of the content and/or recommendations may be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device. At step 918, the method 900 may include monitoring enhancement or maintenance of the user's emotional state, mental state, or a combination thereof. In certain embodiments, for example, the system 100 may track interactions with the application supporting the functionality of the system 100, request further content from the user (e.g., new images of the user, videos of the user, audio of the user, etc.), request new self-assessed emotional states from the user, and utilize a plurality of other techniques to monitoring and track the user's progress. In certain embodiments, for example, if the user sends in new images to the application and the user is now smiling and was previously frowning such a change may be evidence that the user's mental state and/or emotional state is improving. In certain embodiments, the monitoring and enhancement may be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device.

At step 920, the method 900 may include training one or more artificial intelligence models, neural networks, and/or systems based on the results of the monitoring, the self-assessed emotional states, the predicted emotional states, the content accessed and experienced by the user, the recommendations followed by the user, or a combination thereof. In certain embodiments, the training may enable the artificial intelligence models to generate predicted emotional states with greater accuracy over time. Additionally, the training may be utilized to identify and/or generate content that enhances and/or maintains emotional states at a faster rate or longer term. Notably, the method 700 may further incorporate any of the features and functionality described for the system 100, any other method disclosed herein, or as otherwise described herein.

The systems and methods disclosed herein may include still further functionality and features. For example, the operative functions of the system 100 and method may be configured to execute on a special-purpose processor specifically configured to carry out the operations provided by the system 100 and method. Notably, the operative features and functionality provided by the system 100 and method may increase the efficiency of computing devices that are being utilized to facilitate the functionality provided by the system 100 and the various methods discloses herein. For example, by training the system 100 over time based on data and/or other information provided and/or generated in the system 100, a reduced amount of computer operations may need to be performed by the devices in the system 100 using the processors and memories of the system 100 than compared to traditional methodologies. In such a context, less processing power needs to be utilized because the processors and memories do not need to be dedicated for processing. As a result, there are substantial savings in the usage of computer resources by utilizing the software, techniques, and algorithms provided in the present disclosure. In certain embodiments, various operative functionality of the system 100 may be configured to execute on one or more graphics processors and/or application specific integrated processors.

Notably, in certain embodiments, various functions and features of the system 100 and methods may operate without any human intervention and may be conducted entirely by computing devices. In certain embodiments, for example, numerous computing devices may interact with devices of the system 100 to provide the functionality supported by the system 100. Additionally, in certain embodiments, the computing devices of the system 100 may operate continuously and without human intervention to reduce the possibility of errors being introduced into the system 100. In certain embodiments, the system 100 and methods may also provide effective computing resource management by utilizing the features and functions described in the present disclosure. For example, in certain embodiments, devices in the system 100 may transmit signals indicating that only a specific quantity of computer processor resources (e.g. processor clock cycles, processor speed, etc.) may be devoted to training the artificial intelligence model(s), generating predictions relating to emotional and/or mental states, generating predictions relating to mental health improvement or regression, generating predictions relating to optimal or ideal activities and/or interactions to present to a user, and/or performing any other operation conducted by the system 100, or any combination thereof. For example, the signal may indicate a number of processor cycles of a processor may be utilized to update and/or train an artificial intelligence model, and/or specify a selected amount of processing power that may be dedicated to generating or any of the operations performed by the system 100. In certain embodiments, a signal indicating the specific amount of computer processor resources or computer memory resources to be utilized for performing an operation of the system 100 may be transmitted from the first and/or second user devices 102, 111 to the various components of the system 100.

In certain embodiments, any device in the system 100 may transmit a signal to a memory device to cause the memory device to only dedicate a selected amount of memory resources to the various operations of the system 100. In certain embodiments, the system 100 and methods may also include transmitting signals to processors and memories to only perform the operative functions of the system 100 and methods at time periods when usage of processing resources and/or memory resources in the system 100 is at a selected value. In certain embodiments, the system 100 and methods may include transmitting signals to the memory devices utilized in the system 100, which indicate which specific sections of the memory should be utilized to store any of the data utilized or generated by the system 100. Notably, the signals transmitted to the processors and memories may be utilized to optimize the usage of computing resources while executing the operations conducted by the system 100. As a result, such functionality provides substantial operational efficiencies and improvements over existing technologies.

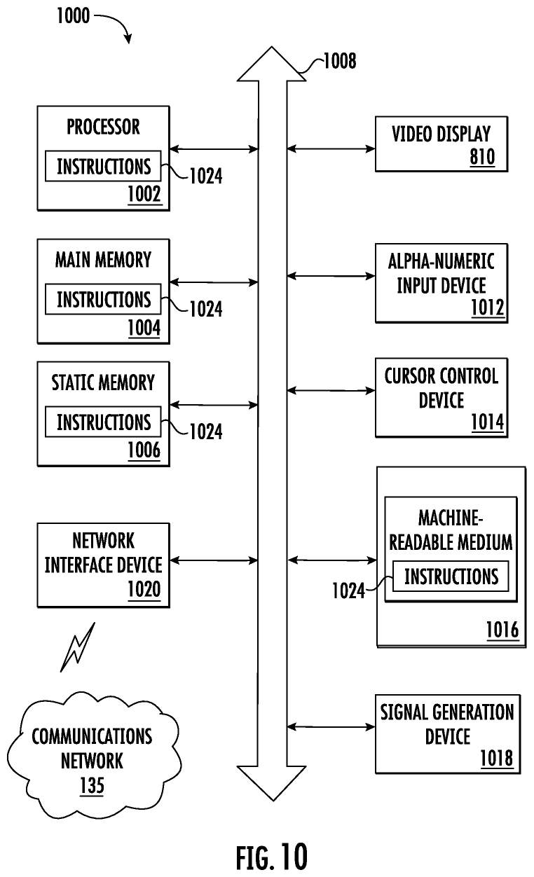

Referring now also to FIG. 10, at least a portion of the methodologies and techniques described with respect to the exemplary embodiments of the system 100 can incorporate a machine, such as, but not limited to, computer system 1000, or other computing device within which a set of instructions, when executed, may cause the machine to perform any one or more of the methodologies or functions discussed above. The machine may be configured to facilitate various operations conducted by the system 100. For example, the machine may be configured to, but is not limited to, assist the system 100 by providing processing power to assist with processing loads experienced in the system 100, by providing storage capacity for storing instructions or data traversing the system 100, or by assisting with any other operations conducted by or within the system 100. As another example, the computer system 1000 may assist with obtaining content associated with physical and/or other attributes of a user, receiving self-assessed emotional states being experienced by the user, extracting features from the content by utilizing any type of artificial intelligence and content processing techniques, determining predicted emotional states of the user based on the extracted features and comparing the features to information utilized to training artificial intelligence models, identifying content to deliver to the user to enhance or maintain an emotional state of the user, generate recommendations for activities to perform to enhance or maintain the emotional state of the user, tracking enhancement and/or maintenance of the emotional state of the user, adapting artificial intelligence models supporting the functionality of the system 100 as inputs and/or data change over time, and/or performing any other operations of the system 100.

In some embodiments, the machine may operate as a standalone device. In some embodiments, the machine may be connected (e.g., using communications network 135, another network, or a combination thereof) to and assist with operations performed by other machines and systems, such as, but not limited to, the first user device 102, the sensor device 107, the second user device 111, the server 140, the server 145, the server 150, the database 155, the server 160, any other system, program, and/or device, or any combination thereof. The machine may be connected with any component in the system 100. In a networked deployment, the machine may operate in the capacity of a server or a client user machine in a server-client user network environment, or as a peer machine in a peer-to-peer (or distributed) network environment. The machine may comprise a server computer, a client user computer, a personal computer (PC), a tablet PC, a laptop computer, a desktop computer, a control system, a network router, switch or bridge, or any machine capable of executing a set of instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein.

The computer system 1000 may include a processor 1002 (e.g., a central processing unit (CPU), a graphics processing unit (GPU, or both), a main memory 1004 and a static memory 1006, which communicate with each other via a bus 1008. The computer system 1000 may further include a video display unit 1010, which may be, but is not limited to, a liquid crystal display (LCD), a flat panel, a solid-state display, or a cathode ray tube (CRT). The computer system 1000 may include an input device 1012, such as, but not limited to, a keyboard, a cursor control device 1014, such as, but not limited to, a mouse, a disk drive unit 1016, a signal generation device 1018, such as, but not limited to, a speaker or remote control, and a network interface device 1020.

The disk drive unit 1016 may include a machine-readable medium 1022 on which is stored one or more sets of instructions 1024, such as, but not limited to, software embodying any one or more of the methodologies or functions described herein, including those methods illustrated above. The instructions 1024 may also reside, completely or at least partially, within the main memory 1004, the static memory 1006, or within the processor 1002, or a combination thereof, during execution thereof by the computer system 1000. The main memory 1004 and the processor 1002 also may constitute machine-readable media.

Dedicated hardware implementations including, but not limited to, application specific integrated circuits, programmable logic arrays and other hardware devices can likewise be constructed to implement the methods described herein. Applications that may include the apparatus and systems of various embodiments broadly include a variety of electronic and computer systems. Some embodiments implement functions in two or more specific interconnected hardware modules or devices with related control and data signals communicated between and through the modules, or as portions of an application-specific integrated circuit. Thus, the example system is applicable to software, firmware, and hardware implementations.

In accordance with various embodiments of the present disclosure, the methods described herein are intended for operation as software programs running on a computer processor. Furthermore, software implementations can include, but not limited to, distributed processing or component/object distributed processing, parallel processing, or virtual machine processing can also be constructed to implement the methods described herein.

The present disclosure contemplates a machine-readable medium 1022 containing instructions 1024 so that a device connected to the communications network 135, another network, or a combination thereof, can send or receive voice, video or data, and communicate over the communications network 135, another network, or a combination thereof, using the instructions. The instructions 1024 may further be transmitted or received over the communications network 135, another network, or a combination thereof, via the network interface device 1020.

While the machine-readable medium 1022 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that causes the machine to perform any one or more of the methodologies of the present disclosure.

The terms “machine-readable medium,” “machine-readable device,” or “computer-readable device” shall accordingly be taken to include, but not be limited to: memory devices, solid-state memories such as a memory card or other package that houses one or more read-only (non-volatile) memories, random access memories, or other re-writable (volatile) memories; magneto-optical or optical medium such as a disk or tape; or other self-contained information archive or set of archives is considered a distribution medium equivalent to a tangible storage medium. The “machine-readable medium,” “machine-readable device,” or “computer-readable device” may be non-transitory, and, in certain embodiments, may not include a wave or signal per se. Accordingly, the disclosure is considered to include any one or more of a machine-readable medium or a distribution medium, as listed herein and including art-recognized equivalents and successor media, in which the software implementations herein are stored.

The illustrations of arrangements described herein are intended to provide a general understanding of the structure of various embodiments, and they are not intended to serve as a complete description of all the elements and features of apparatus and systems that might make use of the structures described herein. Other arrangements may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

Thus, although specific arrangements have been illustrated and described herein, it should be appreciated that any arrangement calculated to achieve the same purpose may be substituted for the specific arrangement shown. This disclosure is intended to cover any and all adaptations or variations of various embodiments and arrangements of the invention. Combinations of the above arrangements, and other arrangements not specifically described herein, will be apparent to those of skill in the art upon reviewing the above description. Therefore, it is intended that the disclosure is not limited to the particular arrangement(s) disclosed as the best mode contemplated for carrying out this invention, but that the invention will include all embodiments and arrangements falling within the scope of the appended claims.

The foregoing is provided for purposes of illustrating, explaining, and describing embodiments of this invention. Modifications and adaptations to these embodiments will be apparent to those skilled in the art and may be made without departing from the scope or spirit of this invention. Upon reviewing the aforementioned embodiments, it would be evident to an artisan with ordinary skill in the art that said embodiments can be modified, reduced, or enhanced without departing from the scope and spirit of the claims described below.

We claim:

1. A system, comprising:
a memory that stores instructions; and
a processor that executes the instructions to configure the processor to:
receive, via a device, content associated with at least one physical attribute, at least one expression, or a combination thereof, of a user, wherein the content associated with the at least one physical attribute, the at least one expression, or a combination thereof, is obtained via at least one sensor;
receive, from the user, at least one self-assessed emotional state currently being experienced by the user;
extract, by utilizing at least one artificial intelligence model, at least one feature from the content;
determine, based on the content and by utilizing at least one artificial intelligence model, at least one predicted emotional state of the user, wherein the at least one predicted emotional state of the user is determined based on comparing the at least one feature extracted from the content to training information utilized to train the at least one artificial intelligence model;
identify, by utilizing the at least one artificial intelligence model, content to deliver to the user to enhance or maintain the at least one self-assessed emotional state, the at least one predicted emotional state, or a combination thereof; and
provide, to the device, access to the content to facilitate enhancement or maintenance of the at least one self-assessed emotional state, the at least one predicted emotional state, or a combination thereof.

2. The system of claim 1, wherein the processor is further configured to determine, by utilizing the at least one artificial intelligence model, whether a deviation between the at least one self-assessed emotional state and the at least one predicted emotional state of the user exists.

3. The system of claim 2, wherein the processor is further configured to train the artificial intelligence model to facilitate a prediction for a future emotional state of the user, another user, or a combination thereof, based on the deviation if the deviation between the at least one self-assessed emotional state and the at least one predicted emotional state of the user is determined to exist.

4. The system of claim 1, wherein the processor is further configured to determine the at least one predicted emotional state of the user based on identifying a correlation of the at least one feature with a pattern in the training information corresponding to at least one known emotional state.

5. The system of claim 1, wherein the processor is further configured to determine whether the at least one predicted emotional state or the at least one self-assessed emotional state has a higher probability of being at least one actual emotional state of the user.

6. The system of claim 1, wherein the processor is further configured to select the at least one predicted emotional state as the at least one actual emotional state for the user if the at least one predicted emotional state has the higher probability of being the at least one actual emotional state of the user.

7. The system of claim 1, wherein the processor is further configured to determine a score value relating to a mental health of the user based on analyzing a plurality of signals associated with a mood, a mental state, or a combination thereof, associated with the user, interaction data associated with the user, or a combination thereof.

8. The system of claim 7, wherein the processor is further configured to determine a deviation between the score value relating to the mental health of the user and the at least one predicted emotional state, the at least one self-assessed emotional state, at least one actual emotional state, or a combination thereof.

9. The system of claim 1, wherein the processor is further configured to receive additional information associated with the user, wherein the additional information comprises a plurality of markers associated with the user, wherein the plurality of markers comprise location information, demographic information, psychographic information, life event information, emotional action information, movement information, health information, audio information, virtual reality information, augmented reality information, time-related information, physical activity information, mental activity information, diet information, experience information, sociocultural information, political information, relationship information, or a combination thereof.

10. The system of claim 1, wherein the content associated with the at least one physical attribute, the at least one expression, or a combination thereof obtained via the at least one sensor comprises image content, video content, audio content, haptic content, vibration content, blood pressure data, sweat data, heart rate data, breath data, breathing data, glucose data, gesture data, motion data, speed data, orientation data, or a combination thereof.

11. The system of claim 10, wherein the video content indicates at least one facial expression, at least one facial movement, or a combination thereof, and wherein the audio content indicates a rate of speech, a tone of the user, a pitch of the user, a volume of speech of the user, or a combination thereof.

12. The system of claim 1, wherein the processor is further configured to combine at least one first characteristic of the at least one self-assessed emotional state with at least one second characteristic of the at least one predicted emotional state to define at least one actual emotional state of the user.

13. The system of claim 1, wherein the at least one self-assessed emotional state identifies an emotional state of the user as expressed in the content.

14. A method, comprising:
receiving, via an application executing on a device, content associated with at least one physical attribute of a user, at least one expression, or a combination thereof, wherein the content associated with the at least one physical attribute, the at least one expression, or a combination thereof, is obtained via at least one sensor;
receiving, from the user via the application, at least one self-assessed emotional state currently being experienced by the user;
extracting, by utilizing at least one artificial intelligence model, at least one feature from the content;
determining, based on the content and by utilizing at least one artificial intelligence model executed by a processor, at least one predicted emotional state of the user, wherein the at least one predicted emotional state of the user is determined based on comparing the at least one feature extracted from the content to training information utilized to train the at least one artificial intelligence model;
generating, by utilizing the at least one artificial intelligence model, content to deliver to the user to enhance or maintain the at least one self-assessed emotional state, the at least one predicted emotional state, or a combination thereof; and
providing, to the device, access to the content to facilitate enhancement or maintenance of the at least one self-assessed emotional state, the at least one predicted emotional state, or a combination thereof.

15. The method of claim 14, further comprising prompting the user to identify the at least one self-assessed emotional state within the content obtained via the at least one sensor associated with device.

16. The method of claim 14, further comprising determining a type of content to deliver to the user to enhance or maintain the at least one self-assessed emotional state, the at least one predicted emotional state, or a combination thereof.

17. The method of claim 14, further comprising providing an option, via the application, to enable the user to provide information reflecting on the self-assessed emotional state, the at least one predicted emotional state, an enhancement of the predicted or self-assessed emotional state, or a combination thereof.

18. The method of claim 14, further comprising generating a recommendation for an activity for the user to perform to facilitate enhancement or maintenance of the at least one self-assessed emotional state, the at least one predicted emotional state, or a combination thereof.

19. The method of claim 14, further comprising requesting the user to generate, for the application, baseline content and identify at least one actual emotional state of the user as represented by the baseline content.

20. A computer-readable device comprising instructions, which, when loaded and executed by a processor, cause the processor to perform operations, the operations comprising:
receiving, via an application executing on a device, content associated with at least one physical attribute of a user, at least one expression, or a combination thereof, wherein the content associated with the at least one physical attribute, the at least one expression, or a combination thereof, is obtained via at least one sensor;
receiving, from the user via the application, at least one self-assessed emotional state currently being experienced by the user;
extracting, by utilizing at least one artificial intelligence model, at least one feature from the content;
determining, based on the content and by utilizing at least one artificial intelligence model, at least one predicted emotional state of the user, wherein the at least one predicted emotional state of the user is determined based on comparing the at least one feature extracted from the content to training information utilized to train the at least one artificial intelligence model;
generating, by utilizing the at least one artificial intelligence model, content to deliver to the user to enhance or maintain the at least one self-assessed emotional state, the at least one predicted emotional state, or a combination thereof; and
providing, to the device, access to the content to facilitate enhancement or maintenance of the at least one self-assessed emotional state, the at least one predicted emotional state, or a combination thereof.
